(19) United States
(12) Patent Application Publication
(10) Pub. No.: US 2024/0197709 A1
(43) Pub. Date: Jun. 20, 2024

(54) POTENTIATING THE TUMOR-SELECTIVE EFFECTS OF NQO1-BIOACTIVATABLE AGENT BY USE OF CMET INHIBITOR

(71) Applicant: The Trustees of Indiana University, Bloomington, IN (US)

(72) Inventors: Edward Ayson Motea, Indianapolis, IN (US); Snehal Bhandare, Indianapolis, IN (US)

(21) Appl. No.: 18/541,121

(22) Filed: Dec. 15, 2023

Related U.S. Application Data
(60) Provisional application No. 63/476,290, filed on Dec. 20, 2022.
(60) Provisional application No. 63/479,684, filed on Jan. 12, 2023.

(51) Int. Cl. A61K 31/4545; A61K 31/352; A61P 35/00

(52) U.S. Cl. CPC A61K 31/4545; A61K 31/352; A61P 35/00

(57) ABSTRACT

Methods are provided for treating cancer, particularly non-small cell lung cancers and pancreatic ductal adenocarcinomas, by administering both a sublethal dose of NAD(P)H:quinone oxidoreductase 1 (NQO1) bioactivatable drug and a sublethal dose of mesenchymal-epithelial transition factor (cMET) inhibitor to the cancer patient. Treatment of other cancers which overexpress cMET and NQO1 are also provided.

### PRIORITY CLAIM

This application claims priority to U.S. Provisional Application 63/476,290, filed Dec. 20, 2022 and to U.S. Provisional Application 63/479,684, filed Jan. 12, 2023.

### FIELD OF THE INVENTION

This invention relates generally to the field of cancer treatment, specifically to the treatment of cancer cells while causing minimal damage to normal cells through the treatment.

### BACKGROUND

Non-small cell lung cancers (NSCLC) and pancreatic ductal adenocarcinoma (PDA) represent the most lethal types of cancer for men and women in the world. In the United States alone, both cancers claim up to 70% patient lives out of approximately 290,000 people diagnosed annually (American Cancer Society 2018). While these patients have many treatment options, the lack of effective and tumor-selective treatment strategies remains a major problem today. Most chemotherapies used in the clinic to treat cancer are toxic because they target both the cancerous cells of the tumors and fast proliferating normal cells due to lack of predictive biomarkers for targeted therapy. Tumor-selective therapies for NSCLC and PDA tumors are mostly non-existent. Palliative therapies for these cancer types have both short- and long-term toxicities, especially when used at high concentrations due to drug resistance. There is an urgent need to develop innovative treatment strategies to selectively target the cancer cells in NSCLC and PDA patients while sparing the normal tissues and to identify reliable biomarkers for personalized cancer therapy. Aspects of the invention disclosed herein address this need.

### SUMMARY OF THE INVENTION

A first aspect of the invention includes a method of treating cancer by administering a sublethal dose of β-Lapachone (β-lap) in combination with administration of a sublethal dose of a mesenchymal-epithelial transition factor (cMET) inhibitor.

A second aspect of the invention includes method of treating solid tumor cells overexpressing cMET and NAD(P)H:Quinone Oxidoreductase 1 (NQO1).

A third aspect of the invention includes a method of identifying cancers overexpressing cMET and NQO1 for targeted treatment by administering β-lap in combination with administration of a cMET inhibitor.

A first embodiment is a method of treating cancer in a subject in need thereof comprising administering to the subject a NAD(P)H:quinone oxidoreductase 1 (NQO1) bioactivatable drug and administering to the subject a mesenchymal-epithelial transition factor (cMET) inhibitor.

A second embodiment is a method of treating cancer in a subject wherein the cancer cells of the subject exhibit increased mRNA expression of the NQO1 gene.

A third embodiment is a method of treating cancer in a subject wherein the cancer cells of the subject overexpress cMET.

A fourth embodiment is a method of treating cancer in a subject wherein the cancer cells of the subject overexpress both NQO1 and cMET.

A fifth embodiment is a method of treating cancer in a subject wherein the cancer cells of the subject are in the form of a solid tumor.

A sixth embodiment is a method of treating cancer in a subject wherein the cancer is recalcitrant.

A seventh embodiment is a method of treating cancer in a subject wherein the cancer is metastatic.

An eighth embodiment is a method of treating cancer in a subject wherein the subject has non-small cell lung cancer (NSCLC) or pancreatic ductal adenocarcinoma (PDA).

A ninth embodiment is use of an NQO1 bioactivatable drug in a therapeutic for the treatment of cancer wherein the NQO1 bioactivatable drug is selected from the group consisting of: β-lapachone (β-lap), β-lapachone (β-lap) analogue ARQ761, deoxynyboquinone (DNQ), DNQ derivative isobutyl-DNQ, napabucasin, streptonigrin, and Akt inhibitor KP372-1.

A tenth embodiment is the use of a NQO1 bioactivatable drug in a therapeutic for the treatment of cancer wherein the NQO1 bioactivatable drug is β-lapachone (β-lap).

An eleventh embodiment is a use of a cMET inhibitor in a therapeutic for the treatment of cancer wherein the cMET inhibitor is selected from the group consisting of: crizotinib and cabozantinib.

A twelfth embodiment is a method wherein administering the cMET inhibitor occurs at least two hours prior to administering the NQO1 bioactivatable drug.

A thirteenth embodiment is a method administering the cMET inhibitor occurs simultaneously with administering the NQO1 bioactivatable drug.

A fourteenth embodiment is a method administering the NQO1 bioactivatable drug occurs simultaneously with administering the cMET inhibitor.

A fifteenth embodiment is a method wherein the NQO1 bioactivatable drug is administered more than once.

A sixteenth embodiment is a method wherein the cMET inhibitor is administered more than once.

A seventeenth embodiment is a method wherein the NQO1 bioactivatable drug is administered at a sublethal dosage.

An eighteenth embodiment is a method wherein the cMET inhibitor is administered at a sublethal dosage.

A nineteenth embodiment is a method further comprising measuring a NQO1 activity and/or a cMET activity of the cancer cells in the subject, wherein the NQO1 activity and/or cMET activity are indicative of therapeutic effectiveness.

A twentieth embodiment is a method further comprising administering to the subject a chemotherapeutic agent or an immunotherapeutic agent.

A twenty-first embodiment is a method wherein the subject is undergoing or subsequently undergoes radiotherapy.

### BRIEF DESCRIPTION OF THE FIGURES

FIG. 1A. Graphs of comparative analysis of NQO1, Catalase (CAT) and MET gene expression in lung tumor versus matched normal lung and image of Western blot comparing NQO1, Catalase and cMET protein levels in normal lung fibroblast (IMR90) versus lung cancer (A549 NSCLC) cell line models.

FIG. 1-1A. Scatterplot graph showing correlation of NQO1 and MET mRNA expression in lung cancer (red) versus associated normal tissues (blue).

FIG. 1-1B. Scatterplot graph showing correlation of NQO1 and MET mRNA expression in pancreatic cancer (red) versus associated normal tissues (blue).

FIG. 1-1C. Graph showing high NQO1 expression in lung cancer significantly correlates with poor survival. (*p=0.034)

FIG. 1-1D. Graph showing high MET expression in lung cancer significantly correlates with poor survival. (*p=0.032)

FIG. 1-1E. Graph showing high NQO1 expression in pancreatic cancer significantly correlates with poor survival. (*p=0.015)

FIG. 1-1F. Graph showing high MET expression in pancreatic cancer significantly correlates with poor survival. (**** p=1.2×10-7)

FIG. 1-1G. Graphs showing comparison of NQO1/CAT ratio in normal lung versus lung tumors and comparison of NQO1/CAT ratio in normal pancreas versus pancreatic tumors (**** p<0.0001).

FIG. 1-1H. Images of Western blot comparison of cMET, NQO1, and Catalase protein expression levels in lung tumor (T) versus matched normal (N) lung tissue from patient samples and graphic of quantification shows that cMET and NQO1 are significantly overexpressed in tumors compared to matched normal tissues; whereas, Catalase is significantly overexpressed in normal tissues compared to matched lung tumor tissue (** p<0.01). Actin was used as the loading control.

FIG. 1B. Graph showing long-term cell survival of A549 NSCLC cells, first pre-treated with non-lethal concentration of crizotinib (criz, 2 μM) for 2 h followed by additional treatment with criz±addition of β-lap at various concentrations for another 2 h. Then the cells were grown in fresh complete media for approximately 7 days post-treatment. LC50 values were calculated from long-term cell survival graphs using GraphPad PRISM; ND denotes Not Determined even at the highest concentration presented. All error bars represent the mean±SEM. **** p<0.0001, *** p<0.001, ** p<0.01, *p<0.05.

FIG. 1C. Graph showing long-term cell survival of A549 NSCLC cells, first pre-treated with various concentrations of crizotinib for 2 h followed by additional treatment with criz (at indicated concentrations)±addition of sub-lethal β-lap (3 μM) for another 2 h. Then the cells were grown in fresh complete media for approximately 7 days post-treatment. LC50 values were calculated from long-term cell survival graphs using GraphPad PRISM; ND denotes Not Determined even at the highest concentration presented. All error bars represent the mean±SEM. **** p<0.0001, *** p<0.001, ** p<0.01, *p<0.05.

FIG. 1D. Graph showing long-term cell survival of IMR90 (a normal lung fibroblast cell line) was treated similar to FIG. 1C, for comparison. LC50 values were calculated from long-term cell survival graphs using GraphPad PRISM; ND denotes Not Determined even at the highest concentration presented. All error bars represent the mean±SEM. **** p<0.0001, *** p<0.001, ** p<0.01, *p<0.05.

FIG. 1E. Graph showing Long-term cell survival of CRISPR-Cas9-mediated knock out of NQO1 in A549 NSCLC cell line (A549 NQO1−/−), treated similar to FIG. 1B for comparison. LC50 values were calculated from long-term cell survival graphs using GraphPad PRISM; ND denotes Not Determined even at the highest concentration presented. All error bars represent the mean±SEM. **** p<0.0001, *** p<0.001, ** p<0.01, *p<0.05.

FIG. 1F. Graph showing long-term cell survival of A549 NQO1−/− cells, treated similar to FIG. 1C for comparison. LC50 values were calculated from long-term cell survival graphs using GraphPad PRISM; ND denotes Not Determined even at the highest concentration presented. All error bars represent the mean±SEM. **** p<0.0001, *** p<0.001, ** p<0.01, *p<0.05.

FIG. 1-1I. Graph showing relative long-term survival of A549 NSCLC treated with varying concentrations of cMETi, foretinib, alone or in combination with β-lap. A549 cells were first pre-treated with varying concentrations of foretinib (For) for 2 h following by additional treatment with variable [for]±addition of sub-lethal β-lap (3 μM) for another 2 h. Then the cells were grown in fresh complete media for approximately 7 days post-treatment.

FIG. 1-1J. Graph showing relative long-term survival of Capan2 PDAC treated with varying concentrations of cMETi, crizotinib, alone or in combination with β-lap. Capan2 cells were first pre-treated with varying concentrations of criz for 2 h followed by additional treatment with variable [criz]±addition of sub-lethal β-lap (2 μM) for another 2 h. Then the cells were grown in fresh complete media for approximately 7 days post-treatment.

FIG. 1-1K. Graph showing relative long-term survival of Capan2 PDAC treated with varying concentrations of β-lap alone or in combination with crizotinib. Capan2 PDAC cells were first pre-treated with crizotinib (2 μM) for 2 h followed by additional treatment with [criz, 2 μM]±addition of β-lap at various concentrations for another 2 h. Then the cells were grown in fresh complete media for approximately 7 days post-treatment.

FIG. 1-1L. Graph showing relative long-term survival plot of isogenic MIAPaCa2 NQO1+/+ versus MIAPaCa2 NQO1−/−PDAC cell lines treated with varying concentrations of β-lap alone.

FIG. 1-1M. Graph showing relative long-term survival plot of isogenic MIAPaCa2 NQO1+/+ versus MIAPaCa2 NQO1−/−PDAC cell lines treated with varying concentrations of criz alone or in combination with β-lap. Cells were first pre-treated with varying concentrations of criz for 2 h followed by additional treatment with variable [criz]±addition of sub-lethal β-lap (2 μM) for another 2 h. Then, the cells were grown in fresh complete media for approximately 7 days post-treatment.

FIG. 1G. Bar graph depicting MET mRNA expression in A549 with stable knock down of MET (shMET) and non-targeting control (shSCR) using shRNA technology.

FIG. 1H. Images of Western blots confirming cMET protein expression in A549 shSCR vs shMET. Downstream targets of cMET kinase activity were also probed for further characterization.

FIG. 1I. Graph showing relative long-term survival studies comparing A549 shSCR vs shMET following treatment with varying concentrations of β-lap±DIC (50 μM) for 2 h. DIC indicates Dicoumarol which is a well-known inhibitor of NQO1 activity used to determine NQO1-dependent response. LC50 values were calculated from long-term cell survival graphs using GraphPad PRISM; ND denotes Not Determined even at the highest concentration presented. All error bars represent the mean±SEM. **** p<0.0001, *** p<0.001, ** p<0.01, *p<0.05.

FIG. 1-1M. Graph showing relative long-term survival of MIAPaCa2 NQO1^{+/+} versus MIAPaCa2 NQO1^{-/-} PDAC cell lines treated with varying concentrations of criz alone or in combination with β-lap. Cells were first pretreated with varying concentrations of criz for 2 h followed by additional treatment with variable [criz]±additional of sub-lethal β-lap (2 μM) for another 2 h. Then the cells were grown in fresh complete media for approximately 7 days post-treatment.

FIG. 1-1N. Graph showing relative long-term survival studies in A549 NSCLC with transient MET knockdown (siMET) compared to siSCR control. β-lap was added±DIC (50 μM) for 2 h after RNAi transfection (48 h) with two different siRNA sequences. Western blot confirmation of cMET knockdown is shown. Dicoumarol (DIC) was used to inhibit NQO1 activity to demonstrate NQO1-dependent response.

FIG. 1-10. Table summarizing the calculated LC50 and DER values obtained from long-term survival experiments with β-lap±DIC. Dose-enhancement ratio (DER) greater than 1 is considered a synergistic response.

FIG. 1-1P. Bar graph showing relative cMET mRNA expression in Capan-2 shSCR versus shMET normalized to GAPDH mRNA expression.

FIG. 1-1Q. Images of Western blots confirming stable cMET knockdown by shRNA in Capan2 PDAC cell line. Cells containing non-targeting scrambled shRNA were used as control (shSCR). Downstream targets of cMET kinase activity were included for further cell line characterization.

FIG. 1-1R. Graph showing relative long-term survival in isogenic Capan2 shSCR versus shMET cell lines using the DNA assay. Various concentrations of β-lap were added±DIC (50 μM) for 2 h, and replaced with fresh complete media to allow surviving cells to grow for approximately 7 days.

FIG. 2-1A. Graph depicting detection of cellular hydrogen peroxide (H₂O₂) levels in A549 cells following indicated treatment conditions: cells were first pre-treated with vehicle (DMSO, 0.1%) or crizotinib (1 μM) for 2 h. Growth media was then replaced with fresh media containing β-lap (3 μM) alone, criz alone (1 μM) or combination±DIC (50 μM) and collected at 15 and 120 min to measure H₂O₂ production using the ROS-Glo™ H₂O₂ Assay from Promega (Madison, WI). The assay was performed in three biological replicates. Two-way ANOVA and multiple t-tests were used for statistical analyses using GraphPad PRISM 7.05. *** p<0.001; ns=not significant.

FIG. 2A. Images of Western blots of nuclear versus cytoplasmic fractions from A549 cells treated with vehicle (DMSO, 0.1%) control and indicated concentrations of β-lap±DIC (50 μM) for 2 h. Lamin B1 and tubulin were used as loading controls for nuclear and cytoplasmic fractions, respectively.

FIG. 2B. Bar graph depicting quantification of pY1234-cMET (activated cMET) in the nucleus at indicated treatment conditions.

FIG. 2C. Images of Western blots of co-IP experiments to investigate the ability of pY1234-cMET to interact with PARP1 following treatment with β-lap (3 and 6 μM for 2 h), an agent known to create reactive oxygen species (ROS) in the presence of NQO1 enzymes.

FIG. 2E. Bar graph depicting quantification of nuclear PLA foci number relative to vehicle control.

FIG. 3A. Images of Western blot assessments for PAR formation (PARP activity), γH2AX formation (surrogate marker for DSBs), and pY1234-cMET in A549 cells at indicated treatment conditions for 2 h. H₂O₂-treated A549 cells (500 μM, 15 min) was used as positive control. Molecular weight (kDa) markers are provided on the right of each blot for reference.

FIG. 3B. Bar graph of relative PAR (black bars) and γH2AX (gray bars) formation at indicated treatment conditions for comparison. Bar graphs are expressed as the mean±SEM. **** p<0.0001, *** p<0.001, ** p<0.01, *p<0.05, ns=not significant.

FIG. 3C. Bar graph of relative cMET phosphorylation at indicated treatment conditions for comparison. Bar graphs are expressed as the mean±SEM. **** p<0.0001, *** p<0.001, ** p<0.01, *p<0.05, ns=not significant.

FIG. 3D. Graphical representation of average 53BP1 foci formation per nucleus at indicated treatment conditions: criz (1 μM), β-lap (3 μM), and DIC separately or in combination. Bar graphs are expressed as the mean±SEM. **** p<0.0001, *** p<0.001, ** p<0.01, *p<0.05, ns=not significant.

FIG. 3E Graph showing quantification of DSB formation (% cells with ≥5 53BP1 foci) following exposure of A549 shSCR vs shMET cells to indicated agents for 2 h (in red arrow), washed out and monitored up to 24 h to determine DSB repair kinetics.

FIG. 3G. Representative flow cytometry plots of dual Annexin V-FITC/PI (propidium iodide) staining experiment to detect apoptotic cells following indicated treatments of A549 cells for a total of 4 h (2 h pre-treatment with criz followed by additional 2 h with criz±β-lap). DIC was used to inhibit NQO1 activity. After treatment, cells were allowed to grow in fresh media for 48 h, which were then stained with Annexin V-FITC/PI for flow cytometric analysis.

FIG. 3H. Bar graphs showing quantification of early and late apoptotic cells (%) at indicated treatment conditions. Bar graphs are expressed as the mean±SEM. **** p<0.0001, *** p<0.001, ** p<0.01, *p<0.05, ns=not significant.

FIG. 3-1A Images of Western blot analyses of A549 NSCLC cells treated with vehicle control (DMSO, 0.1%), foretinib (for, 1 μM) alone, β-lap (3 μM) alone, or combination±DIC (50 μM). Briefly, A549 cells were first pre-treated with vehicle control or cMETi, foretinib, for 2 h. After pre-treatment, media was replaced with fresh media containing indicated treatment conditions for another 2 h. Dicoumarol (DIC), a well-known inhibitor of NQO1, was used to demonstrate NQO1-dependent response. The cells were then harvested and processed for Western blot assessment of PAR formation (indicator of PARP activity) and γH2AX (surrogate marker for DSBs) Tubulin was used as loading control. For reference approximate molecular weight markers (in kDa) are provided for each blot.

FIG. 3-1B Bar graph showing quantification of PAR and γH2AX levels in A549 NSCLC at indicated treatment conditions. Densitometry was measured using ImageJ, and bar graph (mean±SEM) was obtained using GraphPad Prism 9. Two-way ANOVA and multiple t-tests were used for statistical analyses. * p<0.05; *** p<0.001; **** p<0.0001; ns=not significant.

FIG. 3-1C Graph showing quantification of γH2AX formation (surrogate marker for DSBs) in A549 NSCLC cells treated with vehicle control, Criz (1 μM), β-Lap (3 μM), or Combination (COMBO)±DIC (50 μM) Scale bars, 25 μm. Bar graph (mean±SEM) was obtained using GraphPad Prism 9. Two-way ANOVA and multiple t-tests were used for statistical analyses, *p<0.05; ** p<0.01; *** p<0.001; **** p<0.0001; ns=not significant.

FIG. 3-1D Graph showing quantification of γH2AX formation (surrogate marker for DSBs) in MIAPaCa2 PDAC cells treated with vehicle control, Criz (2 μM), β-Lap (2 μM), or Combination (COMBO)±DIC (50 μM) Scale bars, 25 μm. Bar graph (mean±SEM) was obtained using GraphPad Prism 9. Two-way ANOVA and multiple t-tests were used for statistical analyses, *p<0.05; *** p<0.001; ns=not significant.

FIG. 3-1E. Graph showing quantification of 53BP1 foci formation (surrogate marker for DSBs) in MIAPaCa2 PDAC cells with specified treatment conditions. Whisker plot representing number of 53BP1 foci/nucleus was obtained using GraphPad Prism 9. At least 100 nuclei per treatment condition was included. Two-way ANOVA and multiple t-tests were used for statistical analyses; **** p<0.0001.

FIG. 4A. Representative images of A549 NSCLC cell migration at indicated treatment conditions at 16 h post-treatment using a Boyden chamber migration assay: 2 h pre-treatment with criz followed by additional 2 h with criz±β-lap. DIC was used to inhibit NQO1 activity.

FIG. 4B. Bar graph showing quantification of cell migration expressed as a percent of control (vehicle). Quantitative data were shown as the mean±SEM. **** p<0.0001, ** p<0.01, *p<0.05, ns=not significant.

FIG. 4C. Representative images of A549 NSCLC cell invasion at indicated treatment conditions at 16 h post-treatment using a matrigel-coated Boyden chamber invasion assay: 2 h pre-treatment with criz followed by additional 2 h with criz±β-lap. DIC was used to inhibit NQO1 activity.

FIG. 4D. Bar graph showing quantification of cell invasion expressed as a percent of control (vehicle). Quantitative data were shown as the mean±SEM. **** p<0.0001, ** p<0.01, *p<0.05, ns=not significant.

FIG. 4E. Images of Western blot assessment of critical factors involved in invasion and migration following indicated treatment of A549 NSCLC: 2 h pre-treatment with criz (or vehicle control) followed by additional 2 h with criz±β-lap. DIC was used to inhibit NQO1 activity.

FIG. 4F. Bar graph showing quantification of E-Cadherin protein expression. Quantitative data were shown as the mean±SEM. **** p<0.0001, ** p<0.01, *p<0.05, ns=not significant.

FIG. 4G. Bar graph showing quantification of Twist protein expression. Quantitative data were shown as the mean±SEM. **** p<0.0001, ** p<0.01, *p<0.05, ns=not significant.

FIG. 4H. Representative images of A549 shSCR versus shMET migration at 16 h following treatments with DMSO-treated control and β-Lap (3 μM) for 2 h. Scale bars, 50 μm;

FIG. 4I. Bar graph showing quantification of cell migration expressed as a percent of control (vehicle). Quantitative data were shown as the mean±SEM. **** p<0.0001, ** p<0.01, *p<0.05, ns=not significant.

FIG. 4J. Representative images of A549 shSCR versus shMET invasion using a matrigel-coated transwell system at 16 h following treatments with DMSO-treated control and β-Lap (3 μM) for 2 h. Scale bars, 50 μm;

FIG. 4K. Bar graph showing quantification of cell invasion expressed as a percent of control (vehicle). Quantitative data were shown as the mean±SEM. **** p<0.0001, ** p<0.01, *p<0.05, ns=not significant.

FIG. 4-1A. Representative images from in vitro wound healing assay showing that A549 cell migration into the cell-free region (black area in high contrast images) is significantly delayed upon depletion of cMET and/or treatment with sub-lethal β-lap (2 h) compared to isogenic A549 shSCR control.

FIG. 4-1B. Bar graph illustrating percentage wound closure at indicated time points in A549 shSCR versus shMET treated with vehicle control or non-lethal β-lap for 2 h. Two-way ANOVA was used for statistical analyses; * p<0.05.

FIG. 4-1C. Western blot analysis of Vimentin protein expression in A549 shSCR versus shMET treated with vehicle control or non-lethal β-lap doses for 2 h.

FIG. 4-1D. Representative images from in vitro transwell migration assay comparing Capan2 shSCR and Capan2 shMET treated with vehicle control or sub-lethal β-lap (2 μM, 2 h). Images were taken at 16 h post-treatment.

FIG. 4-1E. Bar graph illustrating the percentage of Capan2 shSCR vs shMET PDAC cell migration at 16 h post-treatment with vehicle or sub-lethal β-lap (2 μM, 2 h). Two-way ANOVA was used for statistical analyses; * p<0.05; **** p<0.0001.

FIG. 4-1F. Representative images from in vitro transwell invasion assay comparing Capan2 shSCR and Capan-2 shMET treated with vehicle control or sub-lethal β-lap (2 μM, 2 h). Images were taken at 16 h post-treatment.

FIG. 4-1G. Bar graph illustrating the percentage of Capan2 shSCR vs shMET PDAC cell invasion at 16 h post-treatment with vehicle or sub-lethal β-lap (2 μM, 2 h). Two-way ANOVA was used for statistical analyses; * p<0.05; **** p<0.0001.

FIG. 5A. Representative brightfield images of A549 shSCR versus shMET 3D spheroids treated with vehicle control (0.1% DMSO) or β-lap (3 μM, which is sub-lethal in A549 NSCLC cells in 2D long-term survival assays) imaged at indicated times to monitor microtumor spheroid growth. Scale bar=250 μm.

FIG. 5B. Graphical comparison of 3D spheroid (microtumor) growth of A549 shSCR vs shMET at indicated treatments over time relative to initial 3D spheroid area (day 1).

FIG. 5C. Quantification of 3D spheroid viability at day 10 (in panel A) using CellTiter-Glo® 3D Cell Viability Assay.

FIG. 5D. Top Inset: Representative brightfield, green (NIH3T3-GFP), red (Capan2-mCherry), and merged images of 3D spheroid co-culture of Capan2-mCherry (cancer cells) and NIH3T3-GFP (normal cells). 3D co-cultures were exposed to specified treatment conditions and imaged at indicated times to monitor drug response. Scale bar=250 μm. Untreated NIH3T3-GFP 3D spheroid culture by itself (imaged at indicated times) was added for reference.

FIG. 5E. Bar graph of Capan2-mCherry 3D spheroid microtumor growth at specified treatment conditions over time. Asterisk (*) indicates synergistic response in the combination treatment compared to the corresponding individual treatments alone.

FIG. 5-1A. Representative brightfield images of Capan2 shSCR versus shMET 3D spheroids treated with vehicle control (0.1% DMSO) or β-lap (2 μM).

FIG. 5-1B. Bar graph showing quantification of relative Capan2 shSCR versus shMET 3D spheroid viability at day 4 of treatment using CellTiter-Glo® 3D Cell Viability Assay. T-tests and one-way ANOVA were used for statistical analyses; **** p<0.0001

FIG. 5-1C. Western blot analysis comparing protein expression of cMET, NQO1, and Catalase in NIH3T3 fibroblast cell line (normal cell model) versus Capan2 PDAC cell line (model for tumor). Approximate molecular weight markers (in kDa) are included for reference. Actin was used as loading control.

FIG. 5-1D. Bar graph showing quantification of relative Capan2 3D spheroid viability at day 4 of indicated treatment conditions using CellTiter-Glo® 3D Cell Viability Assay. T-tests and one-way ANOVA were used for statistical analyses; **** p<0.0001; 8 denotes synergistic response to the combination treatment compared to the corresponding individual treatments.

FIGS. 6A and B. Illustration comparing tumor cell and normal cells. In tumors, NQO1 and cMET are aberrantly overexpressed. Bioactivation of β-lap by NQO1 generates ROS—H₂O₂ that promotes cMET activation and translocation from the cell membrane to the nucleus where activated cMET tyrosine kinase interacts with PARP1 for enhanced DNA repair and survival. This is inhibited in the presence of cMET inhibitor, which potentiates β-lap induced DNA damage by compromising PARP1 activity that consequently increases formation of lethal DSBs and cell death. Overexpression of catalase in normal cells provides crucial cellular protection by neutralizing H₂O₂ (if any).

### DETAILED DESCRIPTION

For purposes of promoting an understanding of the principles of the novel technology, reference will be made to embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates.

We have identified critical changes (NQO1 and cMET) in cancer versus normal cells that we can exploit to specifically target the cancer cells for death and predict the right patients for treatment. NAD(P)H:Quinone Oxidoreductase 1 (NQO1) is specifically over-expressed in approximately 90% of NSCLC cells and PDA cells versus associated normal tissues (Bey, et al., Proc Natl Acad Sci USA 2007; 104:11832-7; Bey, et al., Mol Cancer Ther 2013; 12:2110-20; Chakrabarti, et al., Cancer Metabolism 2015; 3:12; Huang, et al., Cancer Cell 2016; 30:940-52; and Motea, et al., Am Assoc for Cancer Res 2019:doi: 10.1158/078-0432.CCR-18-2560). In contrast, we have identified that Catalase (CAT), which degrades hydrogen peroxide (H₂O₂), is concomitantly low (3- to 10-fold) in NSCLC and PDA tumor cells versus associated normal tissues. Normal cells are protected by lack of NQO1 expression and enhanced H₂O₂ scavenging due to relatively high CAT levels. Thus, high NQO1:CAT ratios in tumor cells versus low NQO1:CAT ratios in normal cells represent an exploitable therapeutic window.

β-Lapachone (β-lap) is a unique NQO1-bioactivatable agent that undergoes a futile NQO1-mediated redox cycle that generates >300 M reactive oxygen species (ROS, e.g., H₂O₂) specifically in NQO1+ cancer cells. β-lap induces tumor-selective cell death by creating extensive DNA base damage (e.g., 8-oxoguanine) and single-strand breaks (SSBs) that trigger loss of NAD+ and ATP due to Poly(ADP-ribose) Polymerase (PARP) hyperactivation (Moore, et al., Cell Death & Disease 2015; 6:e1599). β-lap is currently in clinical trials as ARQ761, ClinicalTrials.gov: NCT01502800, NCT02514031. However, clinical trials of β-lap monotherapy show methemoglobinemia and haemolysis due to nonspecific targets at high doses (Gerber et al., J Clin Oncology 2017; 35:2517). Thus, a novel strategy to increase tumor-selectivity was required to potentiate the effects of β-lap at lower doses.

Mesenchymal-epithelial transition factor (cMET) is a proto-oncogene that stimulates cell scattering, invasion, protections from apoptosis and angiogenesis in many cancers including NSCLC and PDA (Maulik et al., Cytokine Growth Factor Rev 2002; 13:41-59; Galland, et al., Cytogenet Cell Genet 1992; 60:114-6; Bottaro, et al., Science 1991; 251:802-4; Comoglio, et al., Nat Rev Drug Discov 2008; 7:504-16; Christensen, et al., Cancer Lett 2005; 225:1-26). In the presence of ROS, cMET can translocate into the nucleus to activate PARP1, a critical factor involved in DNA damage response and repair (Du, et al., Nature Medicine 2016; 22:194-201; Gomes, et al., J Biol Chem 2008; 283:4344-51; Matteucci, et al., Carcinogenesis 2009; 30:937-45). While targeting cMET is an attractive strategy for cancer therapy, the overall efficacies of cMET inhibitors (cMETi), such as Crizotinib, are limited due to dose-limiting toxicities and lack of tumor selectivity as a monotherapy.

The invention described herein leverages cMET activity inhibition for tumor-selective synergistic lethality with a low-dose NQO1-bioactivatable drug to create lethal DNA damage that compromises PARP1 DNA repair activity due to lack of cMET function to provide maximal clinical benefit. Through the disclosed method, nontoxic doses of cMETi can convert sub-lethal doses of a NQO1-bioactivatable drug to exposures that create increased ROS-induced double-strand breaks (DSBs) due to compromised PARP1 activity suppressing DNA repair and metastasis in NQO1⁺ NSCLC and PDA.

Unless explicitly stated otherwise or clearly implied otherwise the term ‘about,’ when used in conjunction with a numerical value, means plus or minus 10 percent (%). For example, the term about 1.0 encompasses all values between and including 0.9 to 1.1.

As defined here a non-lethal or sublethal dosage is any drug concentration dose that inhibit or kill less than 50% of cancer cells. This may be referenced in the specific product information or label for a drug as an inhibitory concentration less than 50% (<IC50) or a lethal concentration less than 50% (<LC50 or LC50) or a lethal dose less than 50% (<LD50). Concentrations that kill greater than 50% of cells are considered lethal.

While the embodiments described herein have been directed to non-small cell lung cancer (NSCLC) and pancreatic ductal adenocarcinoma (PDA), those of skill in the art would appreciate that other cancers overexpressing NQO1 will also benefit from the disclosed method. Such other cancers include, but are not limited to, breast cancers including triple-negative breast cancer, cervical cancer, head and neck cancers, ovarian cancer, colon cancer, liver cancer, brain cancers, and pediatric brain cancers such as Atypical Teratoid Rhabdoid Tumor. Additionally, the methods described herein may be particularly useful in treating cancers which are difficult to treat through other means, such as recalcitrant cancers for which the five-year relative survival rate is below 50 percent and/or metastatic cancers that have spread from the point in the body where it started to another part of the body.

As used herein the term “tumor” encompasses a grouping or mass of cancerous cells both as a group as well as the individual cells of the grouping. As used herein, the term “solid tumor” refers to a grouping or mass of cancerous cells that usually does not contain cysts or liquid areas. Overexpression of NQO1 includes mechanisms which increase mRNA expression of the NQO1 gene. This includes gene amplification within the cell as well as mRNA overexpression. Cancer cells overexpressing NQO1 include those that do not exhibit overexpression of the NQO1 protein product. For example, cancer cells homozygous for NQO1*2 or NQO1*2 mutant show no detectable NQO1 protein expression but do show amplification of mRNA expression.

A NAD(P)H:quinone oxidoreductase 1 (NQO1) bioactivatable drug is one capable of generating ROS—H₂O₂. Preferred NQO1 bioactivable drugs that may be useful in the present invention are ones capable of generating ROS-H₂O₂ during the futile cycle. Such preferred NQO1 bioactivatable drugs include β-lapachone (β-lap), β-lapachone (β-lap) analogue ARQ761, deoxynyboquinone (DNQ), DNQ derivative isobutyl-DNQ, napabucasin, streptonigrin, and Akt inhibitor KP372-1. For most cancers with NQO1, the β-lapachone LD50 values average from 3.0-6.0 μM. As used herein, low doses or non-lethal doses of the NAD(P)H:quinone oxidoreductase 1 (NQO1) bioactivatable drug β-lapachone may range from 0.5-4.0 μM.

In human clinical trials, the maximum tolerated dose (MTD) for β-lapachone (β-lap) analogue ARQ-761 is ˜390 mg/m² as a two-hour infusion every other week (Gerber, et al., British J of Cancer 119, 928-936 (2018)). Therefore, a preferred low dose or non-lethal dose in accordance with the present disclosure would be less than 390 mg/m² to prevent dose-limiting toxicity caused by monotherapy at MTD. In mice, it was found that 10-20 mg/kg of β-lap could be used for synergy treatments (Motea, et al., Clin Cancer Res (2019) 25(8):2601-2609). The maximum-tolerated dose (MTD) for β-lap in HPBCD formulation is about 25 mg/kg. Therefore, a preferred low doses or non-lethal doses in accordance with the present disclosure would be less than 25 mg/kg, less than 24 mg/kg, less than 23 mg/kg, less than 22 mg/kg, less than 21 mg/kg, less than 20 mg/kg, less than 19 mg/kg, less than 18 mg/kg, less than 17 mg/kg, less than 16 mg/kg, less than 15 mg/kg, less than 14 mg/kg, less than 13 mg/kg, less than 12 mg/kg, less than 11 mg/kg, less than 10 mg/kg, less than 29 mg/kg, less than 8 mg/kg, less than 7 mg/kg, less than 6 mg/kg or less than 5 mg/kg.

A cMET inhibitor is a compound capable of inhibiting expression of mesenchymal-epithelial transition factor (cMET) receptor tyrosine kinase. Preferred cMET inhibitors that may be useful in the present invention include crizotinib and cabozantinib. Other cMET inhibitors that may be useful in the present invention include foretinib, trivantinib, capmatinib, golvatinib, tepotinib, and merestinib (LY-2801653). As used herein, low doses or non-lethal doses of the cMET inhibitor drug such as criz may range from 1-3 μM. The MTD for crizotinib has been reported as 250 mg twice daily (Clark, J. W., et al, Future Oncology, 2019, https://doi.org/10.2217/fon-2019-0653). Therefore a low dose or non-lethal dose as used herein in combination with NQO1 bioactivatable agents would preferably be less than 250 mg twice daily, less than 225 mg twice daily, less than 200 mg twice daily, less than 175 mg twice daily, less than 150 mg twice daily, less than 125 mg twice daily, less than 100 mg twice daily, less than 75 mg twice daily, less than 50 mg twice daily.

The cMET inhibitor and the NQO1-bioactivatable agent may be administered at the same time or they may be administered sequentially. When administered sequentially, it is preferable that the cMET inhibitor is administered to the patient prior to the administration of the NQO1-bioactivatable agent. The likely mechanism of DNA damage formation for the disclosed combination treatment is due to replication-induced DNA single-strand breaks and double-strand breaks. This order of administration allows for the inhibition of the repair pathway in the target cells before DNA damage is created, thus ensuring inefficient repair that leads to increased death of the target cells. More DNA damage is formed by several rounds of DNA replication and cell division. When administered sequentially, the period of time between the administrations is preferably at least 2 hours and preferably no more than 72 hours.

As described herein, the administration of the cMET inhibitor and the NQO1-bioactivatable agent according to the method described here results in a synergistic effect upon NQO1⁺ cancer cells. A dose-enhancement ratio (DER) greater than 1 is considered a synergistic response.

The method of administering a cMET inhibitor and a NQO1-bioactivatable agent may also be used in combination with other therapies selectively targeting the solid tumor, such as radiotherapy. Radiation treatment can induce NQO1 expression and could be used as a complimentary therapy after the administration of a cMET inhibitor.

### EXAMPLES

### Example 1

Inhibition of cMET Activity Potentiates the Tumoricidal Effect of Non-Lethal β-Lap in an NQO1-Dependent Manner.

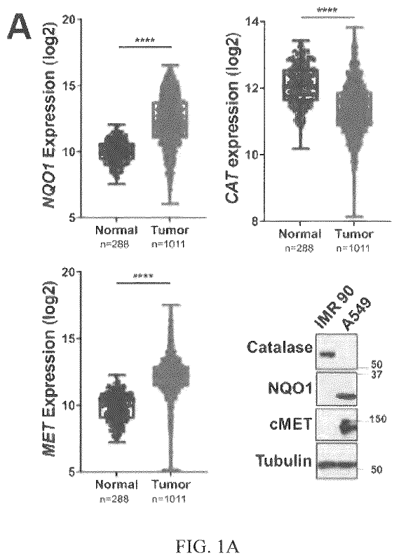

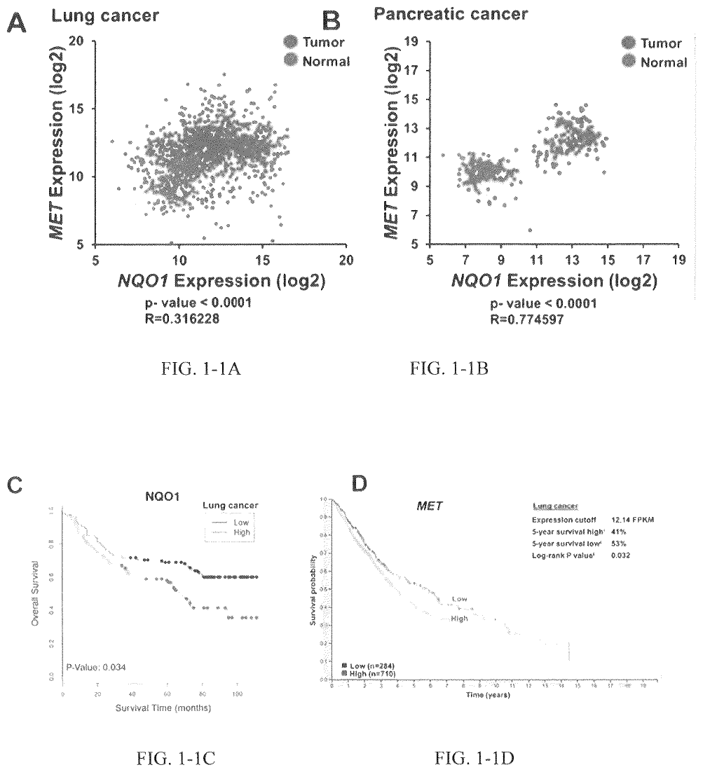

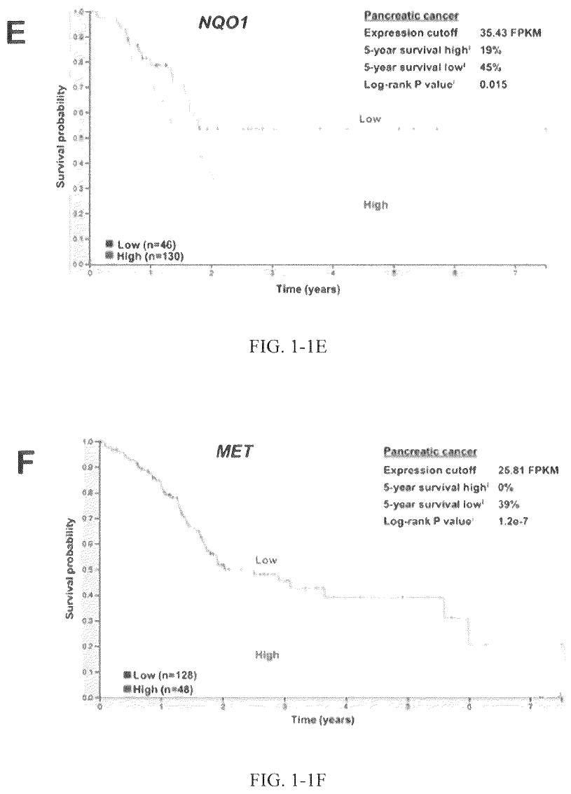

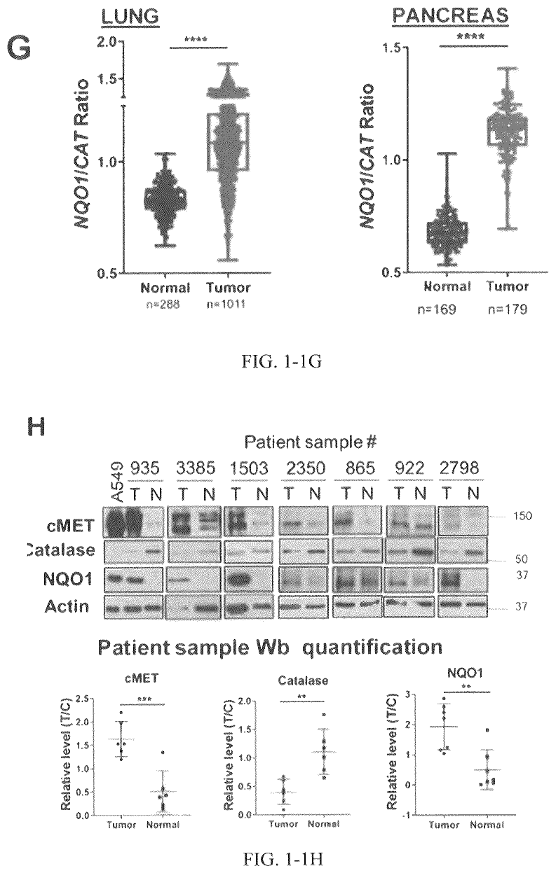

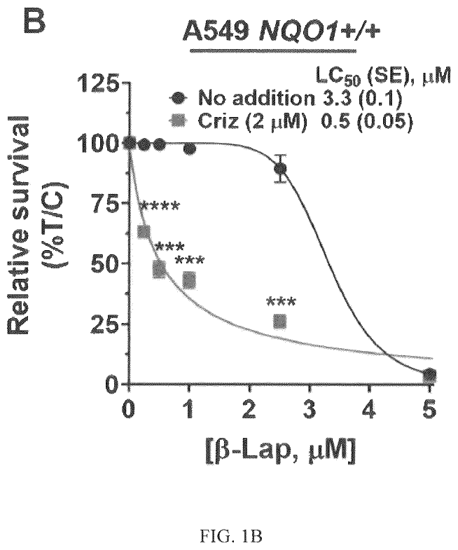

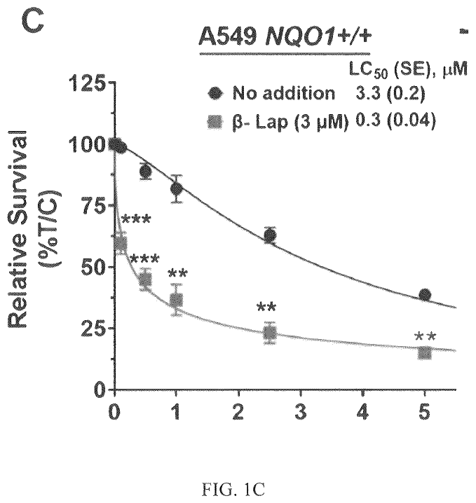

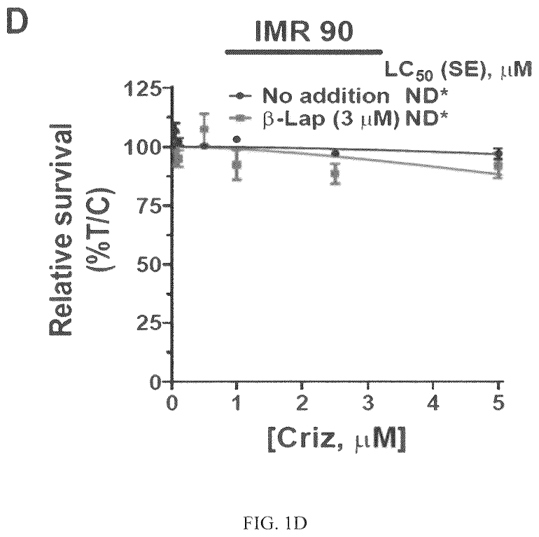

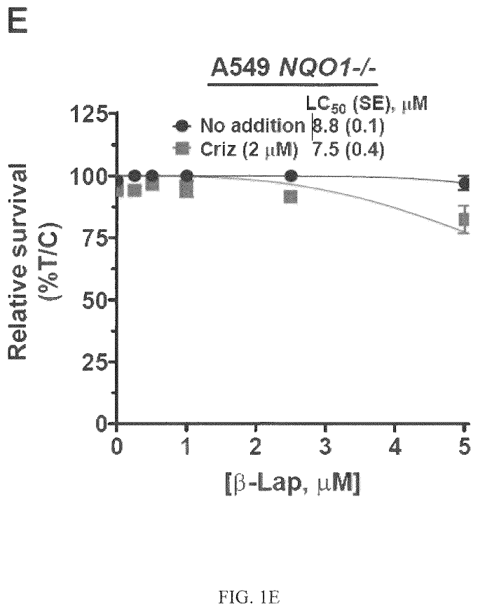

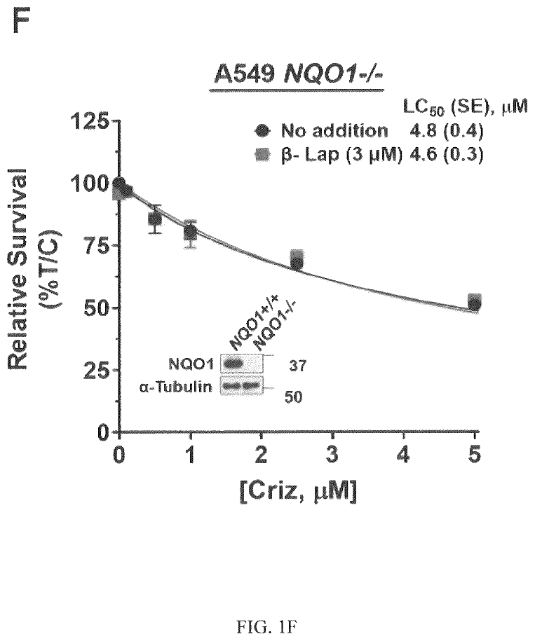

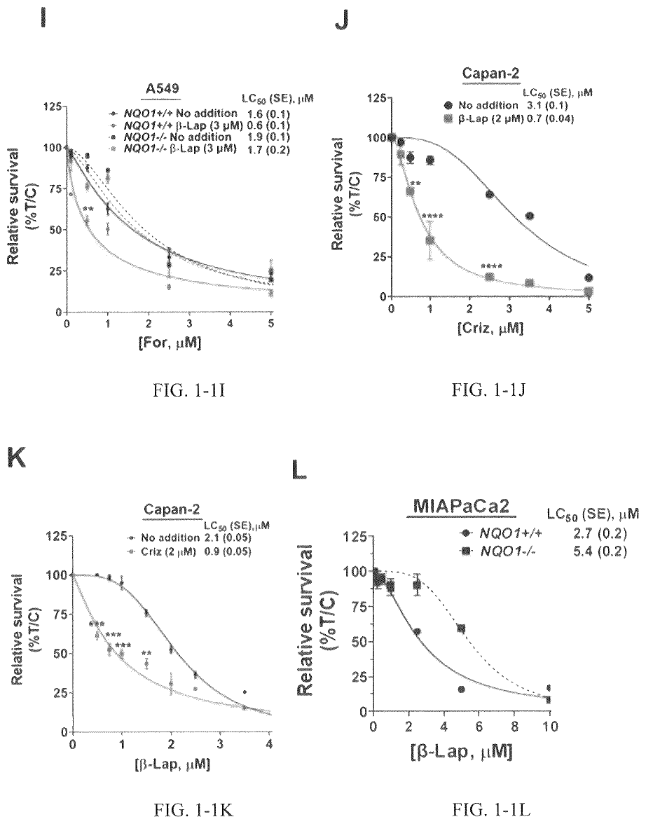

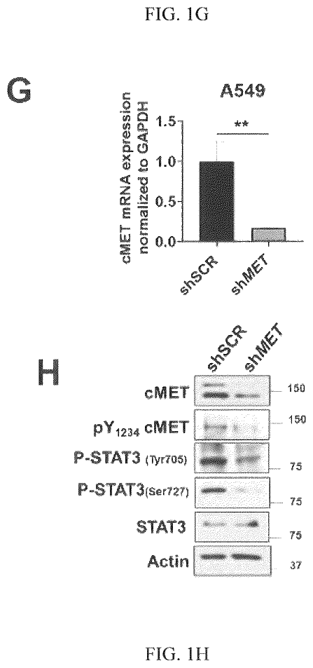

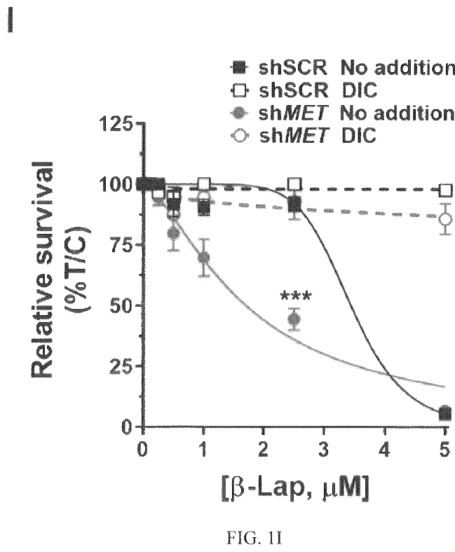

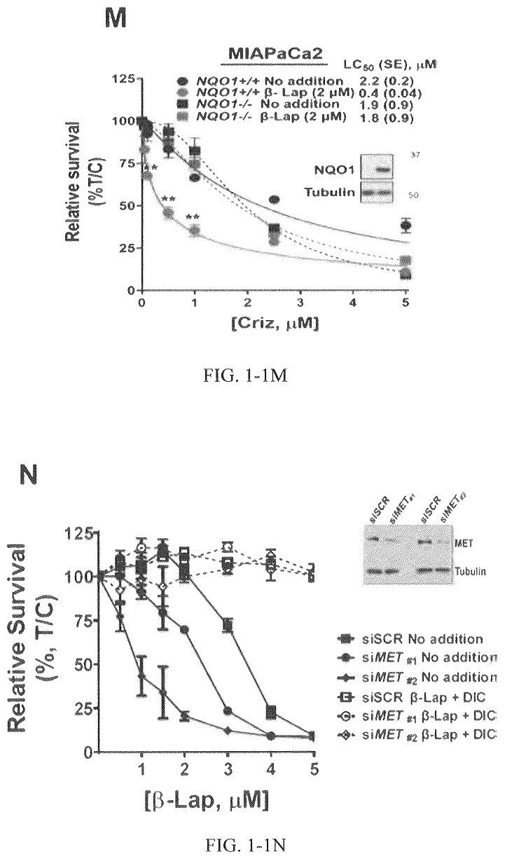

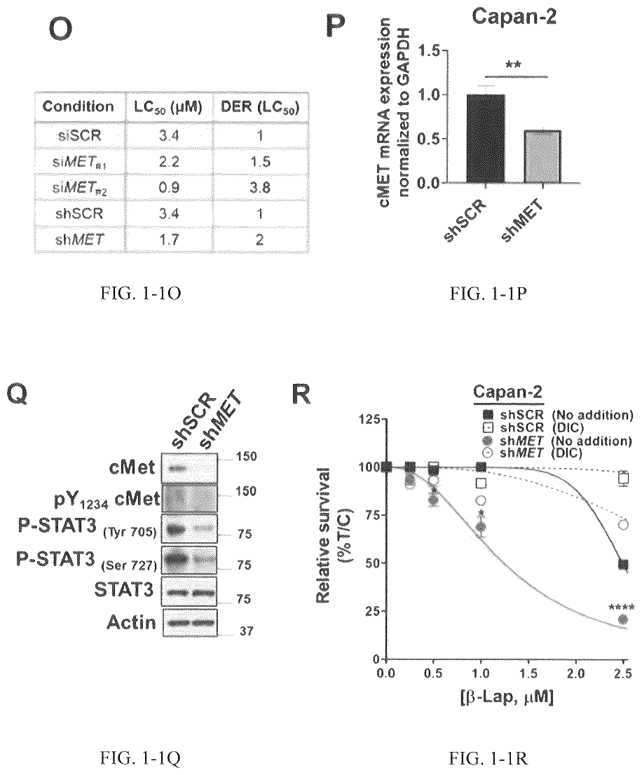

First, the MET, NQO1, and CAT mRNA expression levels were compared in tumor versus associated normal tissues using patient data obtained from The Cancer Genome Atlas (TCGA) (accessible through https://portal.gdc.concer.gov). Our analysis revealed that both MET and NQO1 mRNA levels are significantly overexpressed in lung and pancreatic cancers compared to their corresponding normal tissues (FIG. 1A, FIG. 1-1A, FIG. 1-1B). Moreover, aberrant overexpression of MET or NQO1 in both types of tumors correlated with poor overall patient survival (FIG. 1-1C-F). In contrast, CAT mRNA expression levels are significantly higher in normal versus tumor tissues (FIG. 1A). Elevated NQO1:CAT ratio was noted in patient lung tumor versus paired normal tissues and in pancreatic tumor versus paired normal tissues (**** p<0.0001, FIGS. 1-1G), which offer an exploitable therapeutic window to further increase the tumor selective generation of reactive oxygen species (ROS)—particularly more stable H₂O₂ molecules—created by NQO1-bioactivable drugs in cancers that aberrantly overexpress NQO1. (Singh et al., Cancers (Basel) 2020, 12(4)). High CAT expression in normal tissues provides protection to oxidative damage by converting H₂O₂ into water and oxygen. (De Duve and Baudhuin, Physiol Rev 1966, 46(2), 323-57). Indeed, cMET and NQO1 protein expression levels are significantly higher in A549 (a well-characterized nonsmall cell lung cancer (NSCLC) cell line) compared to normal human lung fibroblast cell line (IMR90) model; whereas, CAT protein levels are higher in IMR90 versus A549 (FIG. 1A). This general trend in protein expression was also observed in the Western blots of primary lung tumor versus paired normal tissue samples from patients (FIG. 1-1H).

cMET has been demonstrated to directly enhance PARP1 activity in DNA damage repair and response to oxidative stress. (Du, et al., Nat Med 2016, 22(2), 194-201). Thus, we posited that aberrant cMET overexpression in cancers provides a critical mechanism of resistance to DNA damage induced by NQO1-bioactivatable agents, especially at sub-lethal doses (<LC50). Therefore, inhibition of amplified cMET activity in cancers could be leveraged to potentiate the tumor-selective ROS-induced therapeutic effect of non-lethal concentration of NQO1-bioactivatable drug by attenuating PARP1 activity toward the formation of irreparable and more lethal damage, consequently causing cell death. To test this, we utilized a long-term cell survival assay to determine the lethality of β-lapachone (β-lap) or crizotinib (criz, an FDA-approved cMET inhibitor) alone or in combination treatments of both agents. Addition of sub-lethal concentration of crizotinib enhanced the sensitivity of A549 to β-lap (DER_{LC50}=6.6, FIG. 1B). Likewise, co-treatment with sub-lethal dose of β-lap enhanced the sensitivity of A549 cells to cMET inhibitors crizotinib (DER_{LC50}=11, FIGS. 1B-C) or foretinib (DER_{LC50}=2.6, FIG. 1-1I). In contrast, normal human IMR90 lung fibroblast cell line that express no NQO1 but with high CAT (FIG. 1A, inset) remained resistant to criz, as well as to combination treatments that are lethal to A549 (FIG. 1D). Other NQO1-overexpressing pancreatic cancer cells, Capan 2 (FIG. 1-1J-K) and MIAPaCa2 (FIG. 1-1L-M) pancreatic ductal adenocarcinoma (PDAC) cell lines, also showed similar hypersensitivity to combination treatment of β-lap+criz compared to either drug alone. Importantly, knocking out NQO1 in A549 NSCLC (FIGS. 1 E-F) and MIAPaCa2 PDAC cell lines (FIGS. 1-1 L-M) rendered them more resistant to β-lap treatment alone (FIGS. 1E, 1-1L) and in combination with cMETi (FIGS. 1E-F, 1-1I, 1-1L-M), highlighting the requirement for NQO1-dependent bioactivation of β-lap for the observed synergistic lethality (vide infra). Together, these results suggest that pharmacological inhibition of cMET activity using cMETi in tumors that overexpress MET and NQO1 potentiates the therapeutic effects of β-lap (ARQ761) at low doses.

Next, we investigated whether loss of cMET activity arising from genetic depletion of MET expression could enhance the therapeutic effect of β-lap at lower concentrations. We utilized the small hairpin RNA (shRNA) technology to stably deplete the expression of MET in A549, and we confirmed reduction of MET mRNA level via RT-qPCR (FIG. 1G) and protein level, including phosphorylation of its downstream target, STAT (Zhang et al., Oncogene 2002, 21(2), 217-26), via Western blot (FIG. 1H). Compared to shSCR control, A549 shMET cells showed hypersensitivity to β-lap treatments (FIG. 1I). Transient knockdown of cMET in A549 using two different siRNAs also displayed sensitization to β-lap treatments compared to siSCR control (FIG. 1-1N-O).

Dicoumarol (DIC) is a well-established NQO1 inhibitor (Asher, et al., Biochemistry 2006, 45(20), 6372-8; Motea, et al., Clin Cancer Res 2019, 25(8), 2601-2609; Huang, et al., Cancer Cell 2016, 30(6), 940-952). Notably, the addition of DIC reversed the lethality of β-lap in isogenic shSCR vs shMET (FIG. 1H) and in siSCR vs siMET (FIG. 1-1N) A549 cell lines. Likewise, stable knockdown of cMET enhanced Capan2 sensitivity to β-lap, which was rescued by DIC (FIG. 1-1P-R). Collectively, these results suggest that compromising aberrant cMET activity in NQO1⁺ cancers is a viable approach to enhance the lethality of NQO1-bioactivatable agents such as β-lap (ARQ761).

### Example 2

NQO1-Mediated Bioactivation of β-Lap Generates ROS-H₂O₂ that Promotes cMET Phosphorylation, Nuclear Translocation and Interaction with PARP1.

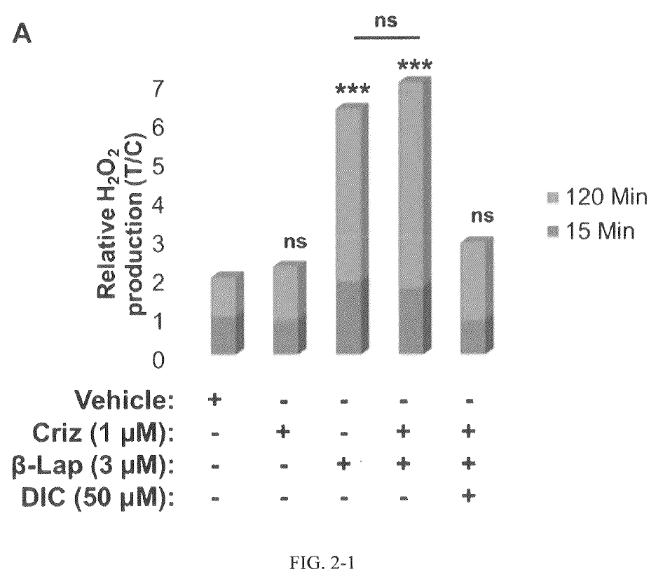

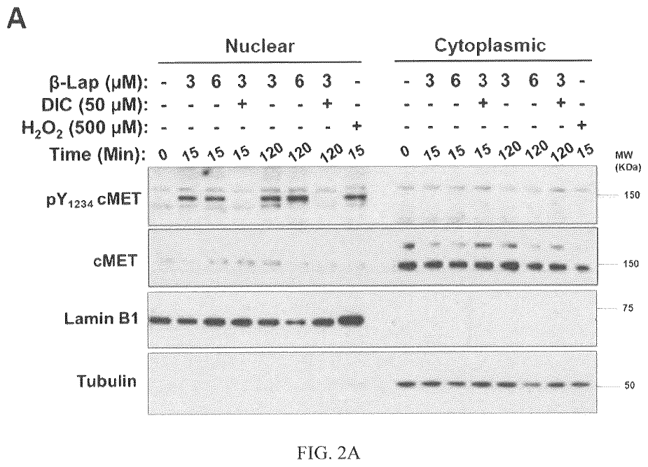

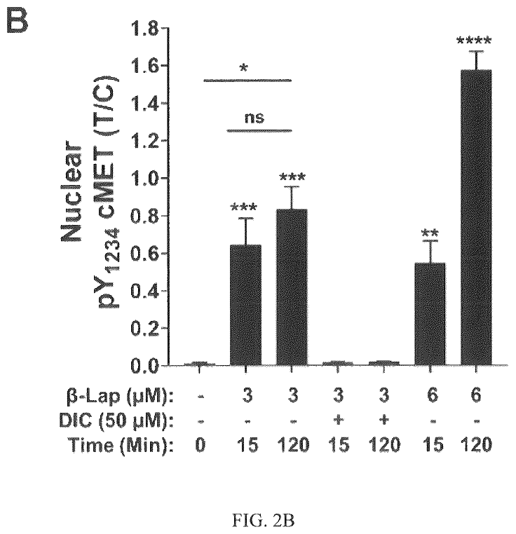

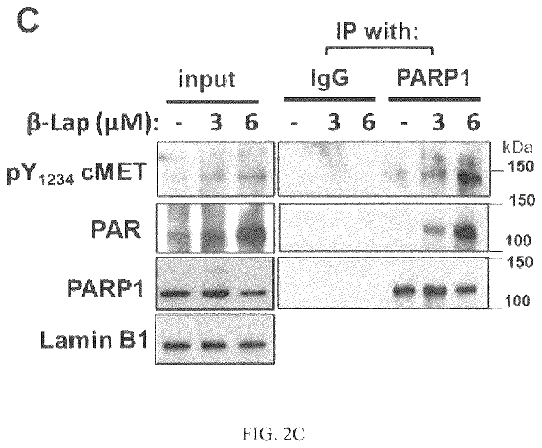

Next, we sought to investigate the mechanistic role of cMET in promoting resistance to ROS-induced DNA lesions caused by the futile redox cycling of β-lap with NQO1. (Pink, et al., J Biol Chem 2000, 275(8), 5416-5424). Consistent with previous studies, we confirmed that exposure of A549 to sub-lethal β-lap (3 μM) significantly increased the relative amount of cellular H₂O₂ within 15 min and further increased at two-hour exposure. (Bey et al., PNAS 2007, 104(28), 11832-11837; Chau, et al., Free Redic Biol Med 1998, 24(4), 660-70). Combination with crizotinib (1 μM) did not alter relative H₂O₂ level, and addition of DIC with β-lap+criz co-treatment prevented H₂O₂ formation (FIG. 2-1A). In response to H₂O₂ stimulation, cMET can translocate into the nucleus where it interacts with PARP1 for activation to enhance the repair of ROS-induced DNA damage. (Chen et al., J Biol Chem 2019, 294(21), 8516-8528). Indeed, we noted that exposure of A549 NSCLC to β-lap (3 μM and 6 μM for 2 h) greatly enhanced the translocation of activated cMET (pY1234-cMET) into the nucleus (FIG. 2A-B) within 15 min and increased at 2 h, which correlated with H₂O₂ formation (FIG. 2-1A). In contrast, addition of DIC with β-lap (3 μM) treatment showed no detectable cMET activation (pY1234-cMET) and nuclear translocation highlighting the requirement for ROS-H₂O₂ generated by NQO1-mediated bioactivation of β-lap. Furthermore, we noted increasing levels of cMET activation (pY1234-cMET) and PAR formation (marker for PARP1 activity) in a β-lap dose dependent manner (FIG. 2C, input). Next we performed PARP1 immunoprecipitation studies to determine whether it associates with activated cMET in the nucleus following treatment with β-lap at increasing doses. Indeed, we co-immunoprecipitated increasing levels of pY1234-cMET with PARP1 at increasing β-lap concentration. We also detected increasing PAR levels coinciding with PARP1 protein (FIG. 2C). Moreover, we detected enhanced in situ interaction between activated cMET and PARP1 (indicated by red PLA foci) in the nucleus following treatment of A549 with β-lap (3 μM, 2 h) as shown by Duolink proximity litigation assay (FIG. 2E). Notably, addition of crizotinib to β-lap decreased pY1234-cMET/PARP1 interaction (relative to vehicle control) suggesting that activated cMET was required for their interaction in response to ROS-H₂O₂. Additionally, addition of DIC to crizotinib+β-lap showed almost similar PLA foci levels as vehicle control further reiterating the role of β-lap-induced ROS-H₂O₂ in stimulating pY1234-cMET/PARP1 nuclear interaction. Taken together, these data suggest that NQO1+ cancer cells treated with β-lap produce a surge of ROS-H₂O₂ that enables cMET activation and translocation into the nucleus where it interacts with PARP1 to enhance DNA repair for survival.

### Example 3

cMET Inhibition in β-Lap-Treated NQO1+ Cancers Promotes Cell Death by Compromising PARP1 Activity that Elicits Increased DSB Formation

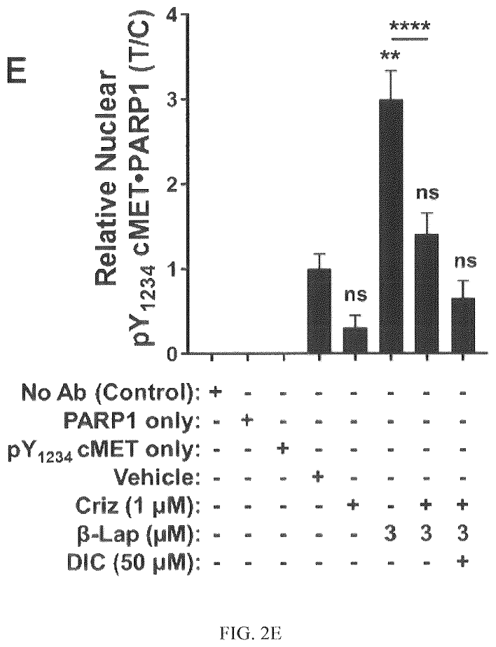

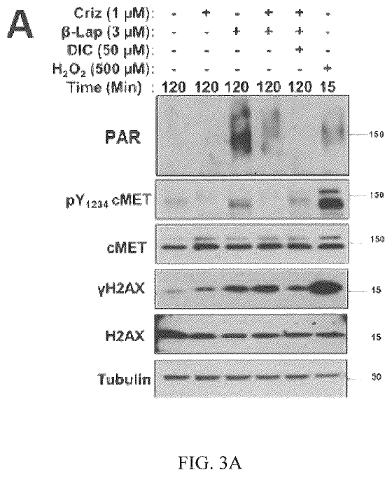

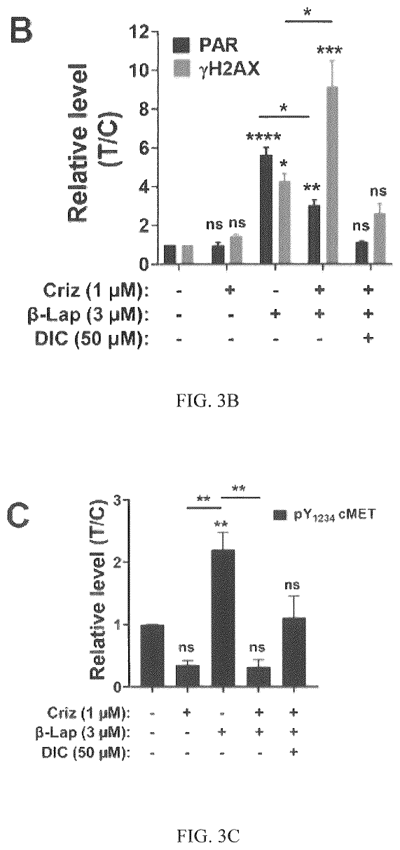

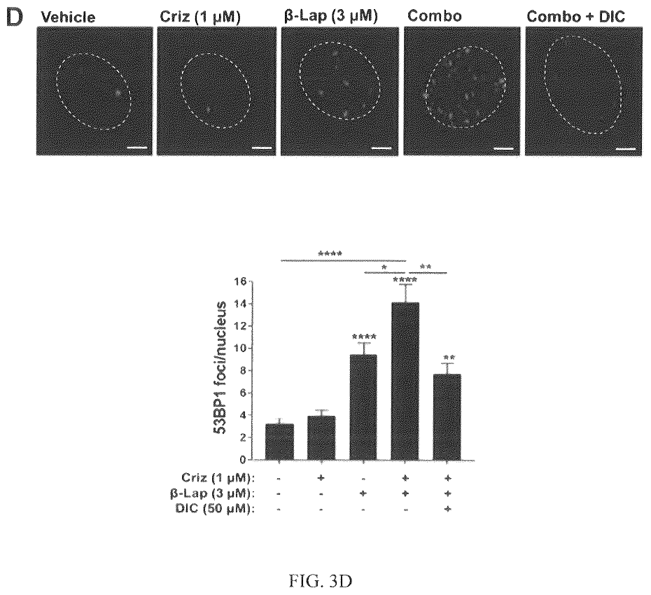

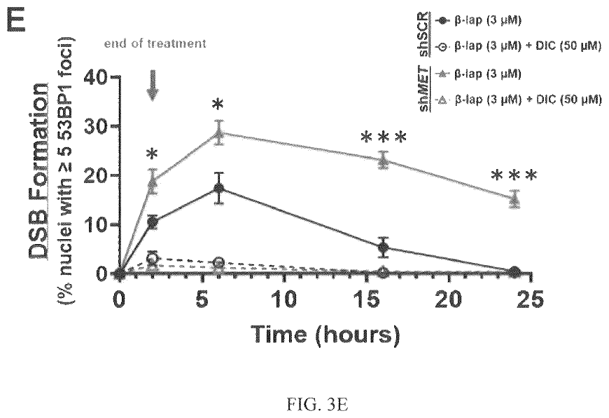

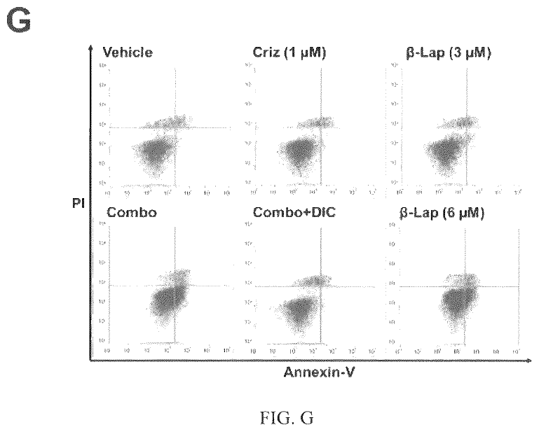

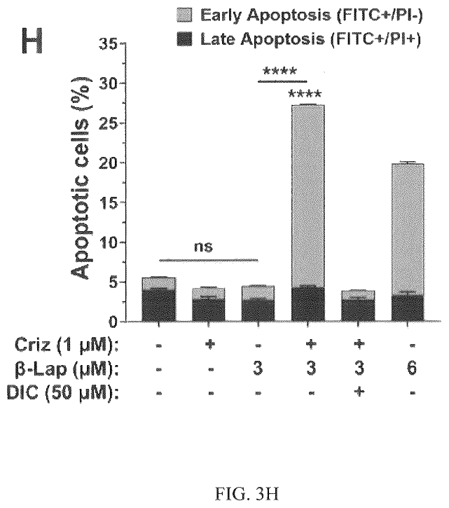

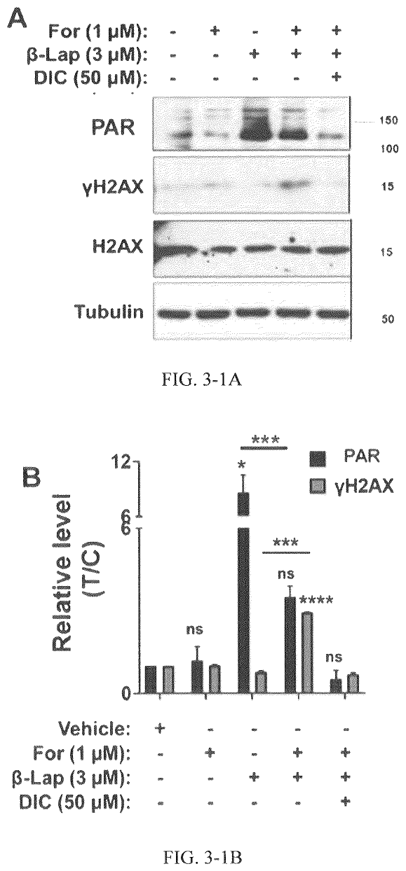

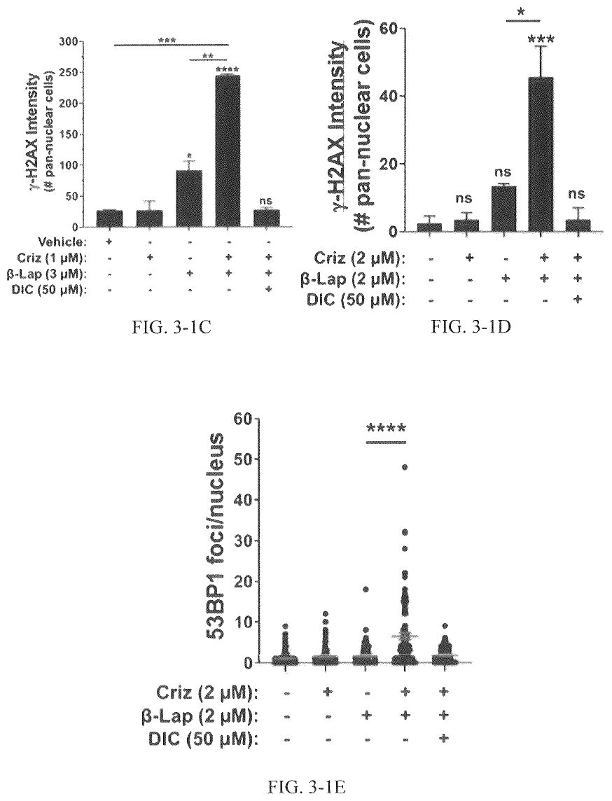

Overexpression of cMET in NQO1+ cancers could provide critical mechanism of survival and drug resistance via enhanced PARP1-mediated repair of DNA damage induced by treatment with β-lap. Thus we investigated whether inhibition of cMET activity in combination with β-lap increased formation of lethal double strand breaks (DSBs) consequently triggering cell death. Using protein immunoblot, we detected more y-H2AX (pS139-H2AX, a surrogate marker for DSBs (Mah, et al., Leukemia 2010, 24(4), 679-86; Celeste, et al., Science 2002, 296(5569), 922-7)) in A549 NSCLC treated with crizotinib+β-lap (FIG. 3A, FIG. 3-1C) or foretinib+β-lap combinations (FIG. 3-1A, 3-1B) at non-lethal concentrations compared to individual treatments alone (FIG. 3A). Using immunofluorescence assays, we also observed more yH2AX formation in A549 NSCLC (FIG. 3-1C) or MIAPaCa2 PDAC (FIG. 3-1D) co-treated with crizotinib+β-lap compared to individual treatments alone. We have previously shown that treatment of A549 cells with sub-lethal β-lap hyperactivates PARP1 (i.e., indicated by PAR formation) and induces DSBs that are easily repaired over time. Here we noted that addition of cMETi to β-lap decreased PAR formation (FIG. 3A-B for crizotinib cMETi; FIG. 3-1A-B for foretinib cMETi) suggesting that attenuation of PARP function by inhibiting cMET activity, (FIG. 3C, a reduction in pY1234-cMET observed compared to β-lap treatment alone) promotes the formation of more DSBs (vide supra) likely due to compromised base-excision and single-stranded DNA break repair of oxidative damage induced by β-lap. We also measured another surrogate marker for DSBs—53BP1 foci formation (Mirman and de Lange, Genes Deve 2020, 34(1-2), 7-23; Panier and Boulton, Nat Rev Mol Cell Biol 2014, 15(1), 7-18)—following treatments of cancer cell lines via immunofluorescence assays (FIG. 3D). Consistent with our prior results, we detected more 53BP1 foci formation in crizotinib+β-lap combination compared to the sum of each individual treatment alone in A549 NSCLC (FIG. 3E) and MIAPaCa2 PDAC cell lines (FIG. 3-1E). Notably, addition of DIC reverted the aforementioned responses observed for the combination treatments to levels almost similar to vehicle-treated control, emphasizing the significance of NQO1 expression in cancers as a predictive biomarker for therapeutic response. Treatment of A549 NSCLC with sub-lethal β-lap (3 μM for 2 h) induced formation of DSBs that were easily repaired over a period of 24 h (FIG. 3E). However, depletion of cMET in A549 cells treated with similar β-lap concentration enhanced the formation of DSBs after 2 h exposure (FIG. 3E), as well as significantly delayed the repair of DSBs (FIG. 3E) suggesting that cMET activity influences tumor response and survival to DNA damage induced by sub-lethal β-lap. Using the dual propidium iodide and Annexin V staining assay to measure apoptotic cells (Conrelissen, et al., Apoptosis 2002, 7(1), 41-7; Vermes, et al., J. Immunol Methods 2000, 243 (1-2), 167-90), we noted that crizotinib+β-lap combination resulted in synergistic amount of apoptotic A549 cells compared to crizotinib or β-lap treatments alone (48 h post-treatments), which both showed similar basal levels of early and late apoptotic cells as the vehicle control (FIG. 3G). Interestingly, the percentage of apoptotic A549 cells resulting from the combination of crizotinib+β-lap was comparable to a supralethal treatment with β-lap alone (6 μM7, FIG. 3G). Inhibition of NQO1 activity with DIC blocked the lethality induced by the combination crizotinib+β-lap treatment (FIG. 3H). Collectively, our data strongly suggest that cMET inhibition thwarts the repair of initial DNA lesions induced by β-lap (e.g., oxidative DNA base damage and single-strand breaks) (Chakrabarti et al., Sci Rep 2015, 5, 17066; Dong, et al., Cancer Res 2010, 70(20), 8088-96; Li, et al, Clin Cancer Res 2011, 17(2), 275-285) by attenuating PARP1 activity, converting initial lesions to more lethal DSBs that trigger cell death in an NQO1-dependent manner.

### Example 4

Depletion of cMET Activity in β-Lap-Treated NQO1⁺ Cancers Inhibit Cell Migration and Invasion

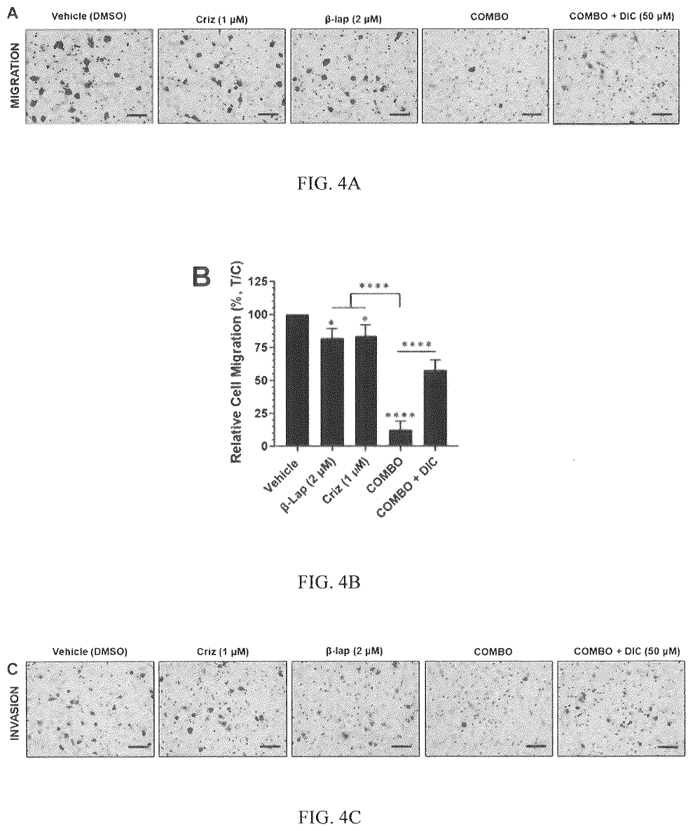

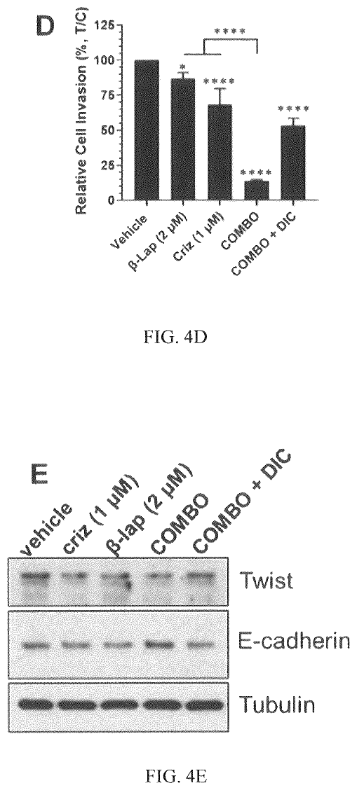

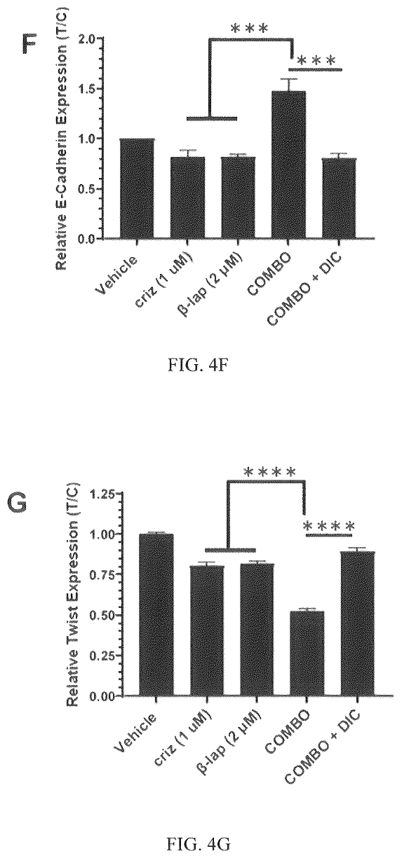

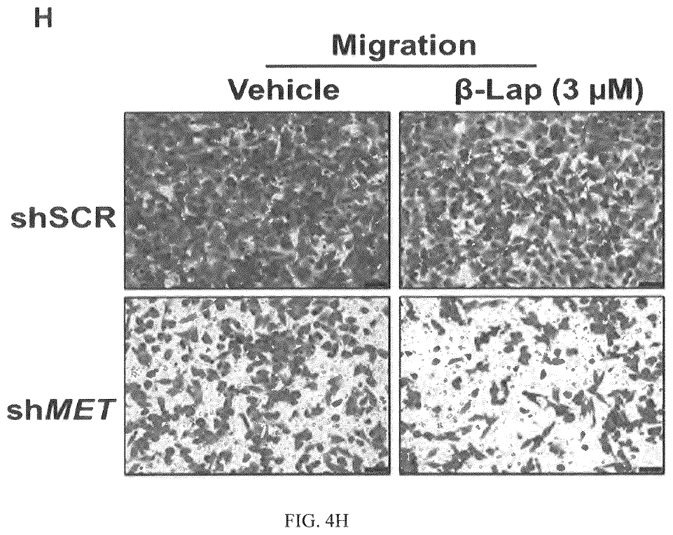

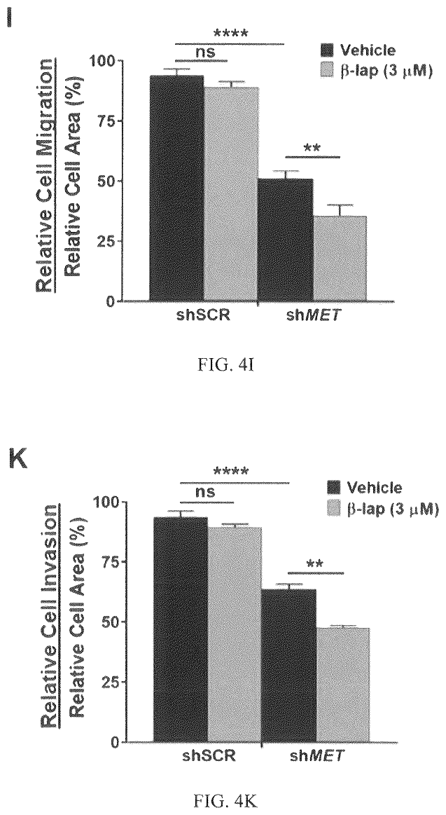

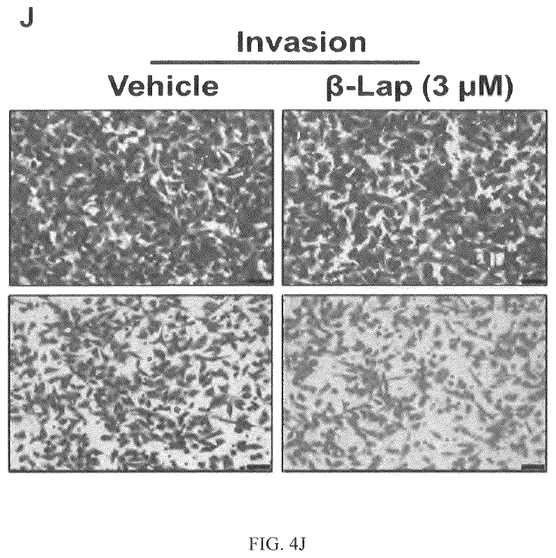

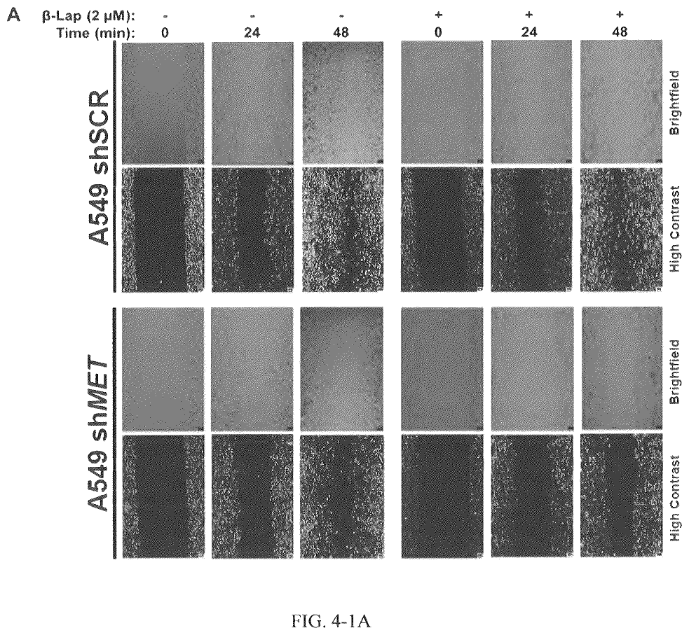

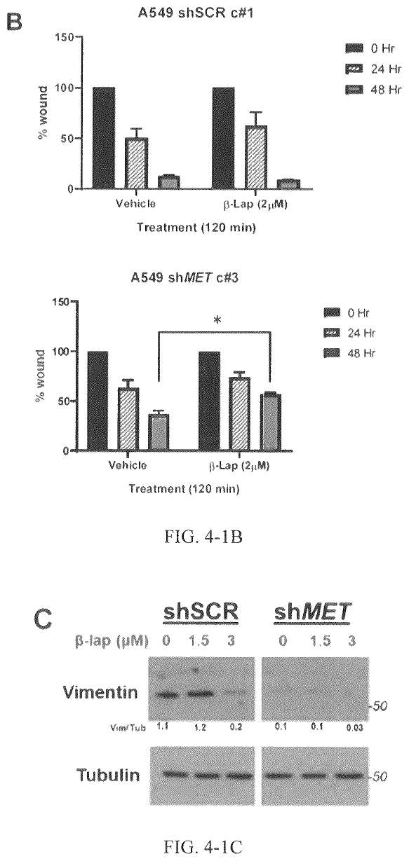

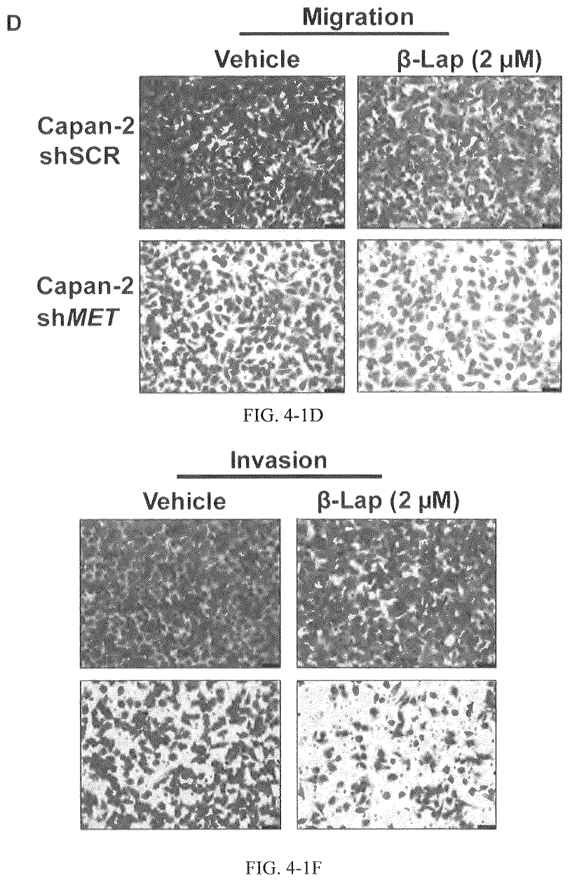

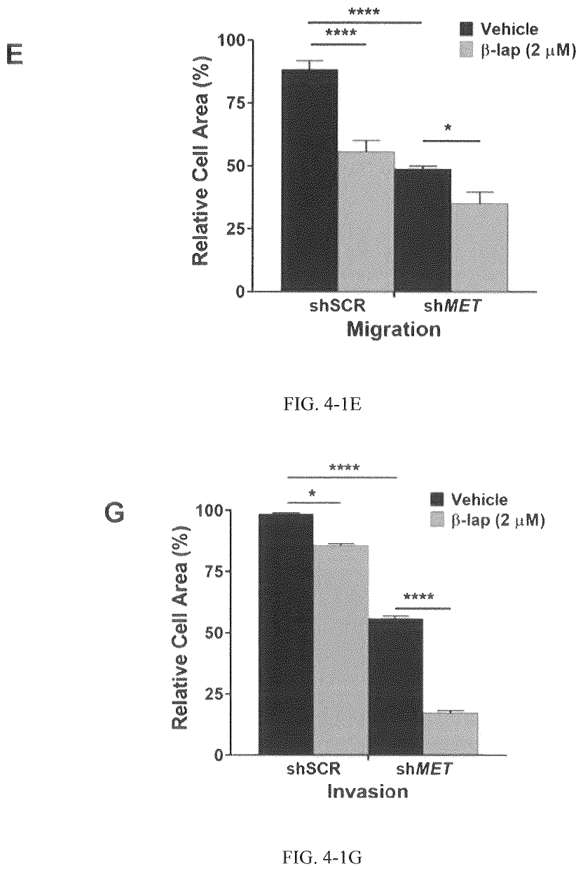

Overexpression of cMET in many cancers, including NSCLC and PDAC, is known to enhance cancer cell mobility, migration, and invasion (Spina, et al., Biomedicines 2015, 3(1), 71-88). NQO1 has also been shown to be consistently overexpressed in highly invasive and metastatic cancers (Li, et al., BMC cancer 2015, 15(1), 207; Madajewski, et al., Mol Cancer Res 2016, 14(1), 14-25). In addition, we noted that lung and pancreatic cancer exhibited significantly higher MET and NQO1 mRNA expression levels compared to their corresponding normal tissues (FIG. 1-1A-B). Thus, we investigated whether treatment with non-lethal β-lap in combination with inhibition of cMET activity (i.e., via pharmacological or genetic interventions) can target and mitigate cancer invasion or migration using Boyden chamber (Chen, Methods Mol Biol 2005, 294, 15-22; Eccles, et al., Biotechnol Annu Rev 2005, 11, 391-421) and wound-healing (Liang, et al., Nat Protoc 2007, 2(2), 329-33) assays. We found that pharmacological inhibition of cMET activity using crizotinib (1 μM)+β-lap (2 μM) combination at non-lethal doses synergistically inhibited the migration (FIG. 4A, B) and invasion (FIG. 4C, D) of A549 cells (16 h post-treatment) compared to the individual treatments alone, which showed slight reductions in migration and invasion. Addition of DIC (NQO1 inhibitor) to the combination treatment restored the ability of A549 to migrate and invade almost to the same level as vehicle-treated control (FIG. 4A-D). In agreement with this, the expression of E-cadherin protein—regarded as a migration and invasion suppressor factor (Beavon, Eur J Cancer 2000, 36 (13 Spec No), 1607-20)—was significantly increased by the end of combination treatment (2 h pre-treatment with crizotinib followed by additional 2 h with crizotinib+β-lap) compared to vehicle-treated control or the individual drugs alone (FIG. 4E-F). Likewise, we noted a decrease in the expression of a transcription factor that promotes invasion and migration, Twist (Khan et al., Tumour Biol 2013, 34(5), 2497-506), with the combination treatment relative to vehicle control (FIG. 4E, G). Addition of DIC to the combination treatment reverted Twist and E-cadherin protein expression to almost similar level as vehicle-treated control. shRNA mediated knockdown of cMET expression in A549 and Capan2 decreased their migratory and invasive phenotypes relative to their corresponding shSCR controls, which is consistent with the canonical role of cMET in invasive cancers. Remarkably, β-lap treatment of A549 with stable genetic knockdown of MET (A549 shMET) significantly reduces cell migration (FIGS. 4H-I, 4-1A-B) and invasion (FIG. 4J-K) compared to β-lap-treated or vehicle-treated A549 shSCR (p<0.001). Indeed, we noted a significant decrease in the expression of Vimentin, a critical factor that induces cell migration and metastasis (Usman, et al., Oncogenesis 2018, 7(2), 21), in A549 with cMET knockdown (shMET) compared to control (shSCR). Notably, treatment of A549 shSCR with sub-lethal β-lap decreased Vimentin expression, which was further reduced in shMET (FIG. 4-1C). Treatment of Capan2 PDAC shMET cells with β-lap also significantly reduced cell migration and invasion compared to isogenic Capan2 PDAC shSCR control in transwell assays (FIG. 4-1D-G). Collectively, these data suggest that cMET and NQO1-addicted tumors could be exploited as biomarkers for targeted therapies with cMETi and NQO1-bioactivatable agents (e.g., β-lap or ARQ761 in clinical trials) to effectively reduce cancer growth, migration and invasion.

### Example 5

Inhibition of cMET Activity with Non-Lethal β-Lap Treatment Potentiates 3D Spheroid Growth Reduction in a Tumor-Selective Manner

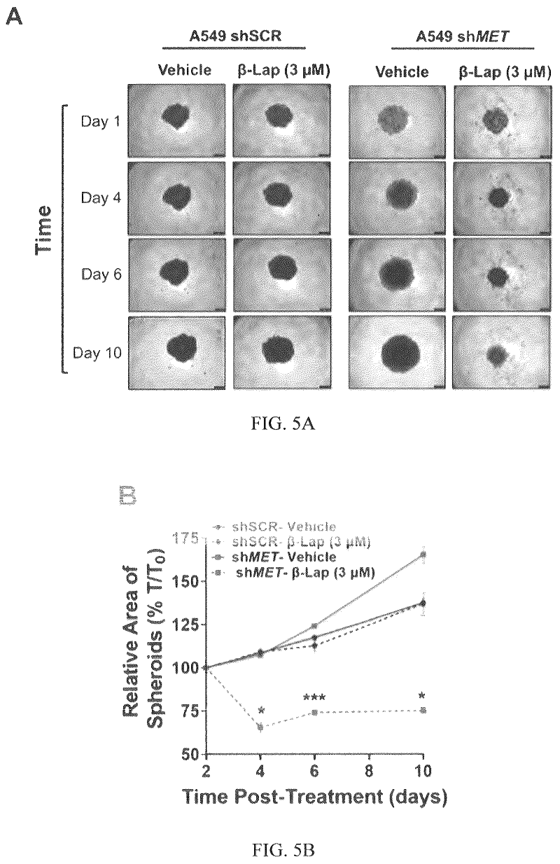

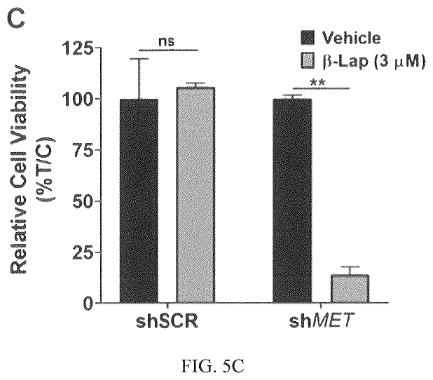

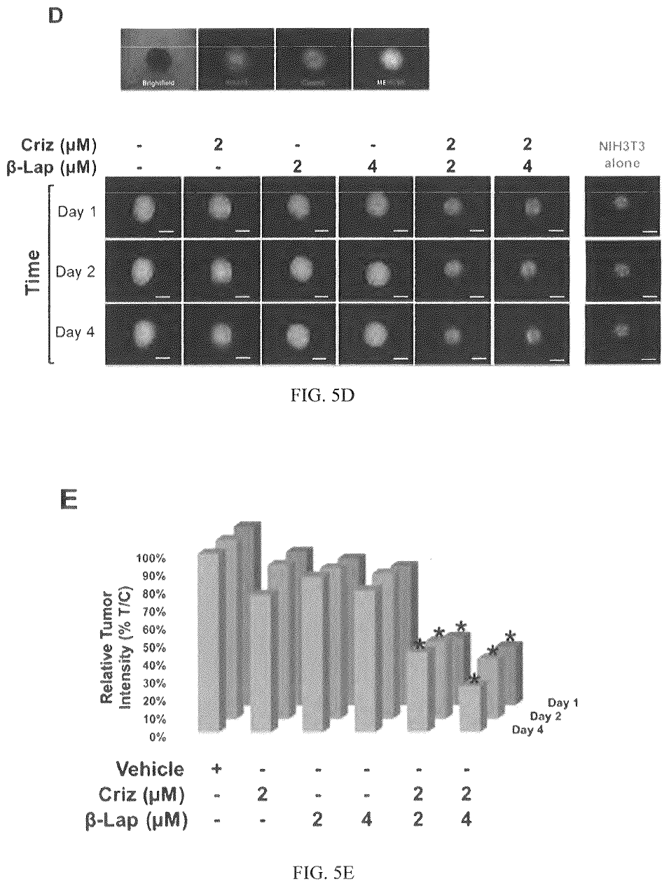

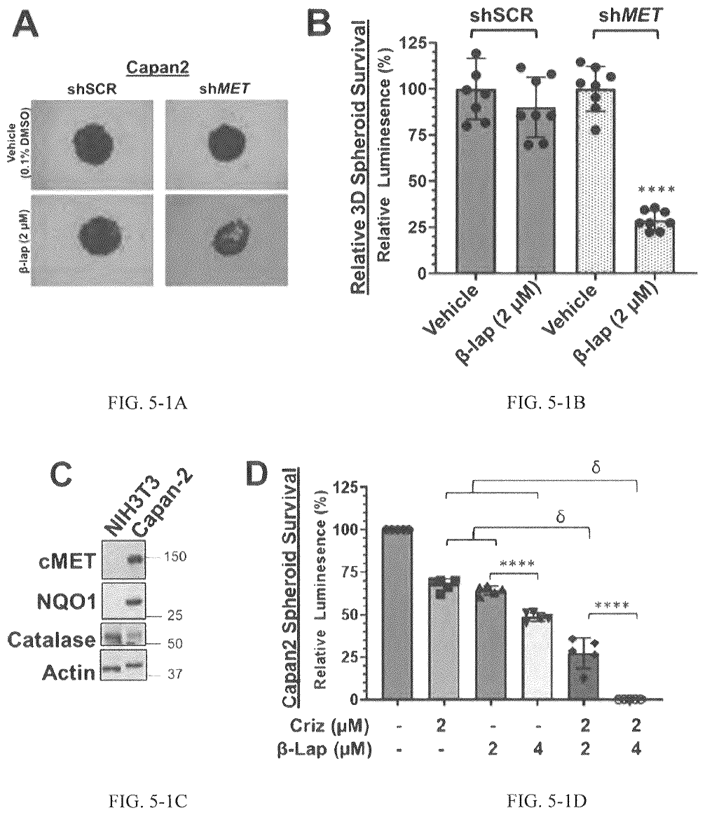

Studies have shown that drug sensitivity of cancer cells cultured in three-dimension (3D) more accurately predicts their sensitivity in vivo.30-31 Thus, we utilized 3D tumor spheroid growth of our isogenic A549 shSCR or shMET cancer cell lines to closely recapitulate the cell-cell interactions of human tumors32-33. After three days of spheroid formation, A549 shSCR and shMET microtumors were exposed to vehicle control (0.1% DMSO) or β-lap (3 μM, sub-lethal concentration in 2D culture for A549 parental line), and imaged over time for 3D spheroid tumor growth for comparison. Remarkably, we noted that genetic depletion of cMET in A549 line (shMET) potentiated the lethal effect of β-lap (3 μM) over time compared to A549 shSCR treated with the same β-lap concentration, which demonstrated relatively negligible microtumor lethality (FIG. 5A-B). Additionally, the viability of A549 shSCR versus shMET 3D spheroids treated with vehicle control and β-lap at day 10 was determined using CellTiter-Glo® 3D Viability Assay (FIG. 5C) for comparison. We found that A549 microtumor with cMET knockdown were more hypersensitive to β-lap treatment compared to A549 shSCR control, which demonstrated no significant lethality. Again, this observation was not limited to A549 NSCLC because we noted similar treatment responses in the viability of isogenic Capan2 shSCR and shMET PDAC 3D spheroids (FIG. 5-1A, 5-1B).

Next, we investigated the tumor-selectivity and efficacy of our combination treatment strategy using 3D co-culture spheroids of Capan2 PDAC (permanently labelled with mCherry) and NIH3T3 mouse embryonic fibroblast cells (permanently labelled with GFP) to mimic physiological cell-cell and cell-environment interactions during treatment. Capan2 PDAC was used as a model for tumor, and NIH3T3 served as a model for normal stromal cells34-35 that have been demonstrated to exhibit phenotypic markers similar to cancer-associated fibroblast cells36. Western blot analysis showed that Capan2 PDAC expressed very high levels of NQO1 and cMET proteins but not in NIH3T3, and Catalase was highly expressed in NIH3T3 compared to Capan2 (FIG. 5-1C). These are consistent with patient data comparing associated normal tissues versus tumors in pancreas (FIGS. 1-1B, 1-1G) and lungs (FIGS. 1A, 1-1B, 1-1H). Using confocal fluorescent microscopy, we noted that NIH3T3-GFP fibroblast cells were surrounding the Capan2-mCherry PDAC cells in the 3D spheroid co-culture (FIG. 5D). Remarkably, treatment of the 3D spheroid co-culture with crizotinib (2 μM)+β-lap (2 μM or 4 μM) induced synergistic and tumor-selective reduction of Capan2-mCherry microtumor growth in a β-lap dose-dependent manner and spared NIH3T3-GFP fibroblast cells even at day 1 of treatment (FIG. 5D-E). Individual treatments with crizotinib (2 μM) or β-lap (2 μM or 4 μM) had very little effect on tumor growth. We observed that as Capan2-mCherry microtumor decreased in mCherry fluorescent signal and surface area in the combination treatments, the surrounding fibroblast NIH3T3-GFP in the spheroid co-culture concomitantly decreased in apparent surface area due to the loss of Capan2-mCherry. Indeed, the overall size and GFP fluorescent intensity of NIH3T3-GFP in the 3D co-culture with the combination treatments were almost comparable to untreated NIH3T3-GFP 3D spheroid alone (FIG. 5D) demonstrating tumor-selective response with the combination treatments. Furthermore, we confirmed synergistic lethality in Capan2 3D spheroids treated with the combination treatments in a β-lap dose-dependent manner compared to the corresponding individual treatments alone via CellTiter-Glo® 3D Viability Assay (FIG. 5-1D).

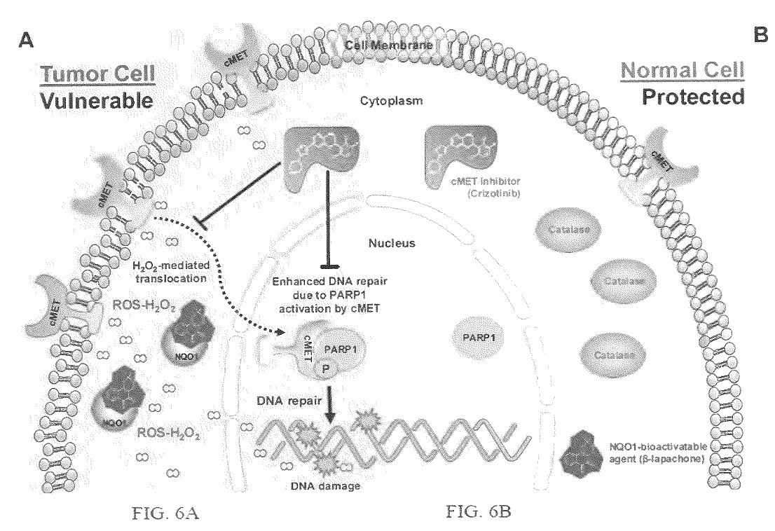

In many solid tumors, overexpression of mesenchymal-epithelial transition factor (cMET) receptor kinase and NADPH Quinone oxidoreductase 1 (NQO1) correlates with poor prognosis. Studies have shown that high cMET and NQO1 expression in cancers promote tumor development, progression, metastasis, and acquired drug resistance. Tumor selective therapies for tumors overexpressing NQO1 and cMET are mostly non-existent, and the overall efficacies of cMET inhibitors and NQO1-bioactivable agents are limited due to dose-limiting toxicities and lack of tumor selectivity at high concentrations as a monotherapy. Collectively, we have shown that overexpression of cMET in cancers that have aberrantly high NQO1 provides critical mechanism of survival and resistance to the DNA-damaging effects of NQO1-bioactivatable agent such as β-lap (ARQ761 in clinical trials), especially at non-lethal doses to prevent host toxicity (FIG. 6A-B). Thus, inhibition of aberrant cMET activity in NQO1+ cancers ameliorates the tumor-selective DNA damage induced by β-lap (at non-lethal) through attenuation of PARP1 activity in BER and SSB repair consequently triggering increased formation of lethal DSBs leading to synergistic cell death (FIG. 6A). Since normal cells have little/no NQO1, there would be little/no H₂O₂ generated with β-lap exposure (thus, no aberrant cMET activation). In addition, overexpression of catalase in normal tissues neutralizes any residual H₂O₂ thereby providing them with very effective protection (FIG. 6B).

### EQUIVALENTS AND SCOPE

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. Those skilled in the art will recognize or be able to ascertain using no more than routine experimentation many equivalents to the specific embodiments described herein. The scope of the present invention is not intended to be limited to the above, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, it is to be understood that the invention encompasses all variations, combinations, and permutations in which one or more limitations, elements, clauses and descriptive terms, from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should be understood that, in general, where the invention, or aspects of the invention is/are referred to as comprising particular elements, features, etc., certain embodiments of the invention or aspects of the invention consist, or consist essentially of, such elements, features, etc. For purposes of simplicity, those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranged can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of the ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5% or up to 1% of a given value. Alternatively, the term can mean within an order of magnitude, for example within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the method of the invention can be excluded from any one or more claims, for any reason, whether or not related to the existence of prior art.

Each of the foregoing patents, patent applications and references are hereby incorporated by reference, particularly for the teaching referenced herein.

I claim:

1. A method of treating cancer in a subject in need thereof comprising:
administering to the subject a NAD(P)H:quinone oxidoreductase 1 (NQO1) bioactivatable drug; and
administering to the subject a mesenchymal-epithelial transition factor (cMET) inhibitor.

2. The method of claim 1, wherein the cancer cells of the subject exhibit increased mRNA expression of the NQO1 gene.

3. The method of claim 1, wherein the cancer cells of the subject overexpress cMET.

4. The method of claim 1, wherein the cancer cells of the subject overexpress both NQO1 and cMET.

5. The method of claim 1, wherein the cancer cells of the subject are in the form of a solid tumor.

6. The method of claim 1, wherein the subject has non-small cell lung cancer (NSCLC) or pancreatic ductal adenocarcinoma (PDA).

7. The method of claim 1, wherein the NQO1 bioactivatable drug is selected from the group consisting of: β-lapachone (β-lap), β-lapachone (β-lap) analogue ARQ761, deoxynyboquinone (DNQ), DNQ derivative isobutyl-DNQ, napabucasin, streptonigrin, and Akt inhibitor KP372-1.

8. The method of claim 9 wherein the NQO1 bioactivatable drug is β-lapachone (β-lap).

9. The method of claim 8 wherein the dose of β-lapachone administered is less than 25 mg/kg.

10. The method of claim 1, wherein the cMET inhibitor is selected from the group consisting of: crizotinib and cabozantinib.

11. The method of claim 10 wherein the dose of crizotinib administered is less than 250 mg twice per day.

12. The method of claim 1, wherein administering the cMET inhibitor occurs at least two hours prior to administering the NQO1 bioactivatable drug.

13. The method of claim 1, administering the cMET inhibitor occurs simultaneously with administering the NQO1 bioactivatable drug.

14. The method of claim 1, wherein the NQO1 bioactivatable drug is administered more than once.

15. The method of claim 1, wherein the cMET inhibitor is administered more than once.

16. The method of claim 1, wherein the NQO1 bioactivatable drug is administered at a sublethal dosage.

17. The method of claim 1, wherein the cMET inhibitor is administered at a sublethal dosage.

18. The method of claim 1, further comprising measuring a NQO1 activity and/or a cMET activity of the cancer cells in the subject, wherein the NQO1 activity and/or cMET activity are indicative of therapeutic effectiveness.

19. The method of claim 1, further comprising administering to the subject a chemotherapeutic agent or an immunotherapeutic agent.

20. The method of claim 1, wherein the subject is undergoing or subsequently undergoes radiotherapy.
